(19) United States
(12) Patent Application Publication
(10) Pub. No.: US 2026/0258363 A1
(43) Pub. Date: Sep. 3, 2026

(54) THERAPY

(71) Applicant: PLASTICELL LTD, Hertfordshire (GB)

(72) Inventors: Aleksandra HABICH-CRAYTON, Hertfordshire (GB); Thomas Patrick WILLIAMS, Surrey (GB); Tatyana PONOMARYOV, Hertfordshire (GB); Giuseppe Alessandro D'AGOSTINO, Hertfordshire (GB); Marina TARUNINA, Hertfordshire (GB); Yen CHOO, Hertfordshire (GB)

(21) Appl. No.: 19/164,423

(22) PCT Filed: Mar. 1, 2024
(86) PCT No.: PCT/GB2024/050560

§ 371 Date: Sep. 11, 2025

(30) Foreign Application Priority Data
Mar. 1, 2023 (GB) 2303026.5

(51) Int. Cl. C12N 5/0789; A61K 35/28

(52) U.S. Cl. CPC C12N 5/0647; A61K 35/28; C12N 2500/46; C12N 2501/13; C12N 2501/2306; C12N 2501/727; C12N 2501/73; C12N 2501/999

(57) ABSTRACT

The present invention relates to a method to expand hematopoietic stem and progenitor cells (HSPC), for use in therapy.

### FIELD OF THE INVENTION

The present invention relates to a methodology for expanding hematopoietic stem and progenitor cells. Also disclosed herein are multipotent cells expanded using the present methodologies, for use in therapy,

### BACKGROUND TO THE INVENTION

Haematopoietic stem and progenitor cell transplantation (HSCT) is the most successful and widely used stem cell therapy to date. HSCT is used to treat conditions where the resident immune system has been compromised, such as in blood disorders or chemo-radiotherapeutic treatment. The use of HSCT is also being clinically proven in gene therapies and is expected to be further extended with new genome editing technologies. Nevertheless, challenges still exist, as the transplants have to be tissue matched to the recipients, making demand higher than supply.

Initially, hematopoietic stem and progenitor cells (HSPC) for transplantation were derived from bone marrow (BM) only. More recently, it was discovered that umbilical cord blood (UCB) also contains HSPC able to engraft in the bone marrow and produce blood cells throughout the lifespan of the recipient. Using UCB as a source of HSPC for use in HSCT has several advantages over more conventional BM; UCB is tested and banked ahead of use and therefore more readily available, UCB also contains more immature stem cells and shows less associated graft versus host disease due to incompatibility of tissue types. However, transplants using cells derived from UCB are limited by the number of cells present in one UCB unit. This quantitative limitation cannot be overcome by the transplantation of multiple UCB units into a single subject because of the predominating engraftment of HSPC from one UCB unit only. Therefore, to date, these transplants have been restricted to use in small children.

Before transplantation, whole cord blood units are firstly separated to discard red blood cells and subsequently the nucleated cells are further enriched by sorting for either CD34+ cells or CD133+ cells. The markers CD133 and CD34 are found amongst numerous progenitor/stem cells including those of the hematopoietic system. Further it has been demonstrated that it is the CD133+ and CD34+ compartment of hematopoietic cells where the long-term repopulating cells reside. Therefore, by increasing the number of these specific cell types it would greatly enhance engraftment and allow UCB HSCT to be applicable for treating older children and adults. Unfortunately using conventional culture methods, Hematopoietic Stem Cells (HSC) characterised by the expression of markers CD133, CD34, CD90 and CD49f, are rapidly depleted as they proliferate and concomitantly differentiate into cell types with restricted potency. As such there has been much interest in developing culture conditions which allow the expansion of these HSC, without compromising their stem cell characteristics.

Several strategies exist to increase the total number of cells in UCB units by trying to mimic the niche or environment where these cells normally reside. In the 1970s it was established that conditions containing serum and specific cytokines, mainly stem cell factor (SCF), thrombopoietin (TPO), interlukin-3 (IL3), interlukin-6 (IL6) and granulocyte colony stimulating factor (G-CSF) could be used for the expansion of HSC in vitro. By the early 90s the first clinical trial using UCB cells expanded in serum-free media containing SCF, G-CSF and MGDF (megakaryocyte growth and development factor) for 10 days, was performed. This expansion method resulted in a 56-fold expansion for the total nucleated cells (TNC) and 4-fold expansion for the CD34+ cells. Patients were infused with one manipulated and one unmanipulated fraction either together or 10 days apart. This trial demonstrated the feasibility of expanding UCB units ex-vivo, and their overall safety. Of the 37 patients treated all showed engraftment, though only 12 were still alive after 30 months. Further work using different combinations of cytokines has led to more defined protocols for in vitro expansion, some of which have shown promise in pre-clinical models. These include the use of SCF, TPO, fms-like tyrosine kinase 3-ligand (FLT3LG), IL3 and IL6, and more recently Wnt1, bone morphogenetic protein 7 (BMP7), angiopoietin-like 5 ANGPTL5 and insulin growth factor binding protein 2 (IGFBP2).

Early observations during the in vitro expansion of hematopoietic cells demonstrated that the accelerated proliferation of cells was associated with a concomitant differentiation of these cells into more committed precursors or more terminally differentiated cells. This led to the hypothesis that the fate commitment is most likely controlled at the epigenetic level, with specific sets of genes being transcribed or silenced at different stages. Hence controlling or altering the epigenome would have consequences on the overall phenotype of the cells and their behaviour. Investigations using histone modifiers including histone deacetylase inhibitors yielded promising results. The first of such studies used 5aza 2′deoxycytidine and trichostatin A. Using this regime UCB CD34+/CD90+ cells expanded 4-fold more than cells expanded on cytokines alone and further retained the ability to repopulate NOD/SCID mice. Extension of these studies to include alternative histone modifying enzymes revealed that other HDAC inhibitors also had similar properties, amongst these valproic acid and scriptaid were particularly effective. This was reported by Chaurasia, P. & Hoffman, R. in “Enriched and expanded human cord blood stem cells for treatment of hematological disorders” (2014), Araki, H. et al. “Expansion of human umbilical cord blood SCID-repopulating cells using chromatin-modifying agents” (2006), and also in WO 2014/189781.

Chemical library screens to find molecules which preferentially allow the expansion of CD34+ cells and prevent their differentiation have yielded several molecules of interest. Of note is the aryl hydrocarbon receptor antagonist StemRegenin1, which is currently being used to expand cells in clinical trials. Another set of compounds of note are the pyrimidoindole derivatives UM729 and UM171, which were found to preferentially expand HSCs with long-term engraftment potential so called long-term HSC (LT-HSC) as determined by the presence of the markers CD34, CD90 (Thy1), and CD49f and the absence of CD38 and CD45RA. Other molecules found to preferentially expand CD34+ cells include resveratrol, GSK-3-inhibitors, p18 protein inhibitors and others.

In particular, the receptor tryosine kinase, RET, is of interest and has been shown to be expressed in murine HSCs. RET plays an important role in their survival in vivo and potentiating outgrowth in vivo when activated by glial-derived neurotrophic factor (GDNF) family ligands and coreceptors, mediating Bcl-2 expression. As reported by Grey et al. in “Activation of the receptor tyrosine kinase RET improves long-term hematopoietic stem cell outgrowth and potency” (2020), RET has been demonstrated to have a role in mediating sustained cellular growth, resistance to stress, and improved cell survival throughout in vitro expansion,

Amifostine, the prodrug of WR1065, was developed as a radioprotectant compound by the US military and approved for clinical use in 1995 under the name Ethyol. The exact mechanisms by which the drug exerts its effects are still being studied, but it is metabolised in vivo into WR1065, a ROS scavenger, that prevents DNA damage through the activation of p53. Studies of the action of this compound on hematopoietic cells in vitro, showed that pre-treatment of bone marrow derived CD34+ cells with WR1065 enhanced the formation of both CFU-GEMM and BFU-E colonies by as much as 38-fold.

WO9625045 discloses thiols including amifostine for haematopoietic stem cell growth.

WO2020/084310 and WO2020/084310 disclose the use of a combination of an aminothiol and an HDAC inhibitor, in combination, to form an expanded population of HSPC, which can subsequently be used in therapy.

EPCR (CD201) has recently emerged as the strongest indicator of LT-HSC engraftment potential. The expression of EPCR has been documented for murine bone marrow. Later, the cells positive for CD201 from the mouse BM and foetal liver were shown to express repopulating potential upon transplantation.

Later, in 2017, it was demonstrated for the first time that a small subset of human cord blood CD34+ cells express endothelial protein C receptor (EPCR/CD201/PROCR) when exposed to the HSC self-renewal agonist UM171. EPCR-positive UM171-treated cells, as opposed to EPCR-negative cells, exhibited robust multilineage repopulation and serial reconstitution ability in immunocompromised mice.

In 2020, it was shown that expanded HSCs phenotypically marked by expression of the stem cell markers CD34, CD90 and EPCR (CD201) are highly enriched for LT-HSCs.

Again in 2020, it was shown that the HSC population CD34⁺EPCR⁺ (CD38/CD45RA)⁻ (simply as EPCR⁺HSCs) have high repopulating and self-renewal abilities, reaching a stem cell frequency of ~1 in 3 cells, the highest described to date. It is therefore desirable to create protocols that result in a greater proportion and number of expanded HSCs expressing EPCR,

### SUMMARY OF THE INVENTION

It is possible to achieve an expansion of HSPC by culturing the cells in the presence of a combination of an HDAC inhibitor, for example scriptaid, and an aminothiol compound such as WR1065. The present invention is based at least in part on data presented herein showing an improvement of three exemplary protocols of the invention (PTC13325.1, PTC13325.2 and PTC13325.3) compared to control and also the protocol disclosed in WO2020/084310 and WO2020/084310 (PTC13303). It is believed that the presence of an additional culturing step, before culturing again in the presence of an HDAC inhibitor and then, subsequently, an aminothiol compound, produces expanded cells wherein the total number of nucleated cells is increased.

It was also surprisingly found that the protocols of the invention result in expanded cells that have a greater proportion of the HSCs expressing the marker EPCR. EPCR is a strong indicator of LT-HSC engraftment potential, and therefore expression of this marker in a greater proportion of the expanded population is a desirable feature, which is particularly important for engraftment and leads to the conclusion that the protocols of the invention result in expanded cells that have superior engraftment potential.

It was further surprisingly found that the protocols of the invention show a greater proportion of early lymphoid and myeloid progenitors in the expanded population of cells (particularly relative to PTC13303). This is a desirable feature in any therapeutic product since platelet and neutrophil recovery is an important short-term indicator of treatment success. Delayed platelet and neutrophil recovery are risky for patients and leads to longer hospital stays.

Therefore, the expanded cells produced according to the methods of the invention can produce therapeutic products that demonstrate both superior engraftment potential and increased platelet and neutrophil recovery in patients.

Therefore, the first aspect of the present invention relates to a method to expand hematopoietic stem and progenitor cells (HSPC) wherein the method comprises:
i) obtaining an isolated population of HSPC;
ii) culturing the isolated population of HSPC for at least 48 hours to form a first cultured population;
iii) adding a histone deacetylase inhibitor (HDAC inhibitor) and further culturing the cells, to form a second cultured population; and
iv) adding an aminothiol compound to the second cultured population and further culturing the cells to form an expanded population of cells, wherein the aminothiol compound has the formula

RNH(CₙH₂ₙ)NH(CₙH₂ₙ)SX, wherein R is hydrogen, an aryl, an acyl, or an alkyl group containing from 1 to 7 carbon atoms, each n has a value of from 2 to 6 and X is H or PO₃H₂; or a pharmaceutically acceptable salt thereof.

A second aspect is a composition comprising an expanded population of cells for use in therapy, wherein the cells have been expanded by the method comprising:
i) obtaining an isolated population of HSPC;
ii) culturing the isolated population of HSPC for at least 48 hours to form a first cultured population;
iii) adding a histone deacetylase inhibitor (HDAC inhibitor) and further culturing the cells, to form a second cultured population; and
iv) adding an aminothiol compound to the second cultured population and further culturing the cells to form an expanded population of cells, wherein the aminothiol compound has the formula

RNH(CₙH₂ₙ) NH(CₙH₂ₙ)SX, wherein R is hydrogen, an aryl, an acyl, or an alkyl group containing from 1 to 7 carbon atoms, each n has a value of from 2 to 6 and X is H or PO₃H₂; or a pharmaceutically acceptable salt thereof.

A third aspect is a method of treatment comprising the steps of:
i) obtaining an isolated population of HSPC;
ii) culturing the isolated population of HSPC for at least 48 hours to form a first cultured population;
iii) adding a histone deacetylase inhibitor (HDAC inhibitor) and further culturing the cells, to form a second cultured population; and
iv) adding an aminothiol compound to the second cultured population and further culturing the cells to form an expanded population of cells, wherein the aminothiol compound has the formula 25

RNH(CₙH₂ₙ)NH(CₙH₂ₙ)SX, wherein R is hydrogen, an aryl, an acyl, or an alkyl group containing from 1 to 7 carbon atoms, each n has a value of from 2 to 6 and X is H or PO₃H₂; or a pharmaceutically acceptable salt thereof.

A fourth aspect is use of a composition comprising an expanded population of cells, in the manufacture of a medicament for use in therapy, wherein the cells have been expanded by the method comprising:
i) obtaining an isolated population of HSPC;
ii) culturing the isolated population of HSPC for at least 48 hours to form a first cultured population;
iii) adding a histone deacetylase inhibitor (HDAC inhibitor) and further culturing the cells, to form a second cultured population; and
iv) adding an aminothiol compound to the second cultured populations and further culturing the cells to form an expanded population of cells, wherein the aminothiol compound has the formula RNH(CₙH₂ₙ)NH(CₙH₂ₙ)SX, wherein R is hydrogen, an aryl, an acyl, or an alkyl group containing from 1 to 7 carbon atoms, each n has a value of from 2 to 6 and X is H or PO₃H₂; or a pharmaceutically acceptable salt thereof,
A fifth aspect is a kit for the expansion of HSPC as defined above, wherein the kit comprises; sterile elements for the expansion of HSPC, a HDAC inhibitor and an aminothiol compound having the formula RNH(CₙH₂ₙ)NH(CₙH₂ₙ)SX, wherein R is hydrogen, an aryl, an acyl, or an alkyl group containing from 1 to 7 carbon atoms, each n has a value of from 2 to 6 and X is H or PO₃H₂; or a pharmaceutically acceptable salt thereof.

A sixth aspect is an expanded population of cells obtainable by the method as described above.

### BRIEF DESCRIPTION OF THE FIGURES

FIG. 1 shows the gating strategy to identify human CB Long Term HSCs by Flow Cytometry.

FIG. 2A shows the fold change in the total number of TNC compared to that of the pre-expanded cells for two independent UCB donors (Donor 1 and Donor 2) after expansion using protocols: Ctrl, PTC13303 and PTC13325.1. The combined data is shown in the left plot.

FIG. 2B shows the estimated percent of Lin−/CD34+ cells after treatment with protocols: Ctrl, PTC13303 and PTC13323.1 for Donor 1 and Donor 2.

FIG. 2C shows the calculated percent of long-term HSC (LT-HSC) cells (defined by markers CD34+/CD38−/CD45RA−/CD90+/CD49f+) within (i) TNC populations or (ii) CD34+ cell populations for Donor 1 and Donor 2.

FIG. 2D shows the total number of LT-HSCs (defined by markers CD34+/CD38−/CD45RA−/CD90+/CD49f+) calculated using TNC numbers and percent of LT-HSC within TNC populations for Donor 1 and Donor 2.

FIG. 2E compares the protocols PTC13303, PTC13325.1 and PTC13325.2 in vitro using UCB from two additional donors (Donors 3 and 4).

FIG. 2F shows the percentage of different populations of hematopoietic progenitor cells after treatment with protocols: Ctrl, PTC13303, PTC13323.1 and PTC13325.2 for Donor 3.

FIG. 3 shows the (i) total number of colonies scored for cells expanded from Donor 1 and Donor 2 and (ii) number of CFU-GEMM colonies correlating with numbers of most primitive HSC, which are capable of multilineage haematopoiesis, and reflects their potency to form colonies of mixed morphology.

FIG. 4A shows (i) the percent of human cells, determined by human CD45 expression in the mouse bone marrow aspiration (BMA) at weeks 10 and 16 after transplantation of expanded cells (each dot represents the percentage of human CD45 cells in the BMA of individual animals) and (ii) human hematopoietic cell lineages within engrafted human CD45-pos cells analysed in the mouse BMA using human lineage specific antibodies.

FIG. 4B shows the (i) percent of human cell engraftment in the mouse Bone Marrow after the animals were sacrificed at Week 18 post transplantation, determined by human specific CD45 antibody, and (ii) percent of human LT-HSC cells (CD34+/CD38−/CD45RA−/CD90+/CD49f+) within the population of engrafted human CD45 cells.

FIG. 5 shows A: UMAP plot of sample density by protocol; B: UMAP of the Leiden clustering assignment; C: dot plot of the cluster markers, with log-normalized expression by color and percentage of cells expressing the gene in cluster by size. The high expression of CD34, SPINK and JUN points to Clusters 1, 4, and 6 as the ones comprised of potential LT-HSCs/HSPCs, whereas clusters 5 and 9 are comprised of cycling HSPCs; D: violin and box and whiskers plot of the U-statistic values for HSC signatures (CD34+, PROCR+, CD38−) showing an enrichment of higher scores for PTC13325.2 compared to the control expansion media and to PTC13303. Box and whiskers plot show median (dark thick bar) and the first and third quartiles (box), with 1.5*the inter-quartile range (whiskers); E: box and whiskers plots of differential abundance analysis as neighbourhood log 2 (fold change) distribution per cluster, coloured by log 2 (fold change) and sized by spatial FDR (sFDR) showing a statistically significant positive enrichment of LT-HSCs/HSPCs in cultures treated with PTC13325.2 compared to the control expansion media. n.s.=non-significant, sFDR>0.05 (GMP: Granulocyte-Monocyte Progenitor; HSPC: Haematopoietic Stem and Progenitor Cell; MKP: MegaKaryocytic Progenitor; MEP: Megakaryocitic/Erythroid Progenitor; MEMP: MegaKaryocytic/Erythroid/Mast cell Progenitor; DC: Dendritic Cell).

FIG. 6A compares the protocols Ctrl, PTC13303 and PTC13325.2 ex vivo using UCB from up to 6 independent donors (n=4-6).

FIG. 6B compares the fold change in the number of primitive engraftable HSCs for protocols Ctrl, PTC13303 and PTC13325.2 ex vivo (n=4-6).

FIG. 6C compares the expression of the phenotypic markers of LT-HSCs, (i) CFD49f and (ii) EPCR by CD34+CD38−CD45RA−CD90+ and CD34+CD38−CD45RA−CD90− cell populations in ex vivo expanded cells for protocols Ctrl, PTC13303 and PTC13325.2.

FIG. 7A compares the protocols Ctrl, RET agonist and PTC13325.3 ex vivo using UCB from up to 6 independent donors (n=4-6).

FIG. 7B compares the fold change in the number of primitive engraftable HSCs for protocols Ctrl, RET agonist and PTC13325.3 ex vivo (n=4-6).

FIG. 7C shows the expression of the phenotypic markers of LT-HSCs, (i) CD49f and (ii) EPCR by CD34+CD38-CD45RA-CD90+ and CD34+CD38-CD45RA-CD90-cell populations in ex vivo expanded cells for protocol PTC13325.3.

FIG. 7D shows an assessment of the numbers of produced total nucleated cells (TNT) versus the numbers of cells in the LT-HSC compartment for protocols Ctrl, PTC13303, PTC13325.2 and PTC13325.3 for 4 independent donors.

FIG. 7E shows (left) the total number of colonies scored for cells expanded ex vivo and (right) the total number of CFU-GEMM colonies scored for cells expanded ex vivo by protocols Ctrl, PTC13303, RET agonist, PTC13325.2 and PTC13325.3 (n=3).

### DETAILED DESCRIPTION OF THE INVENTION

As used herein the term hematopoietic stem and progenitor cells (HSPC) refers to cells found in bone marrow, umbilical cord blood and peripheral blood which can differentiate and/or proliferate to form blood cells, examples of blood cells include, but is not restricted to, monocytes, macrophages, neutrophils, basophils, eosinophils, erythrocytes, dendritic cells, megakaryocytes, platelets, T cells, B cells, and natural killer cells.

As used herein the term “hematopoietic stem cells” or “HSC” refers to multipotent or pluripotent cells which have the ability to differentiate into blood cells of all lineages and to regenerate themselves whilst maintaining their pluripotent characteristics.

As used herein the term “long-term hematopoietic cells (LT-HSC)” refers to HSCs that are capable of self-renewal i.e., LT-HSCs can sustain hematopoietic systems for the life of the animal. LT-HSC's preferably include Lin−, CD34+, CD133+, CD38−, CD45RA−, CD90+, CD49f+ and/or CD201+ (EPCR). Within the terms “CD34+”, “CD133+”, “CD90+”, “CD49f+” the (+) designation indicates that the specified cluster of differentiation (CD) is expressed by the cell and is present on the cell surface. Within the terms “CD38-”, “CD45RA−” the (−) designation indicates that the specified CD is not expressed or poorly expressed by the cell. However, human embryonic stem cells and any cell resulting from the destruction of a human embryo are not within the scope of the invention.

As used herein the term “isolated population” refers to a sample of cells which has been obtained from a source. Wherein the cells may have been obtained commercially, or wherein the cells were obtained from a subject. The source of an isolated population includes, but is not restricted to, umbilical cord blood, bone marrow and peripheral blood. The “isolated population” may have been obtained from a source which is fresh or frozen, wherein a fresh source has not been frozen prior to use. If the sample is frozen then the cells will be thawed before use in the method.

As used herein the term “cultured population” refers to an isolated population of cells which has been propagated in an artificial medium ex vivo. It will be obvious to a skilled person what type of artificial media to use, an example of a suitable media is StemSpan ACF media (Stem Cell Technologies). This is sometimes referred to herein as Base media or Basal media. The artificial media may also be supplemented with other factors or cytokines to improve the growth of the cells, examples of supplements include, but are not restricted to, stem cell factor (SCF), fms-related tyrosine kinase 3-ligand (FLT3LG) and thrombopoietin (TPO). It may also be supplemented with IL-6. The isolated population can be cultured/propagated over a number of days to form a cultured population. In some embodiments, the culturing time is from 2 to 20 days, more preferably 3 to 20, most preferably 4 to 15 days. For example, the culturing/propagating can take place over 20, 15, 10, 9, 8, 7, 6, 5 or 4 days. The total culturing time encompasses the time taken to perform steps (ii), (iii) and (iv).

The term c-Jun N-terminal kinase (JNK) inhibitor as used herein refers to a compound which inhibits the activity of JNKs. JNKs belong to the mitogen-activated protein kinase family and are responsive to stress stimuli, such as cytokines, ultraviolet irradiation, heat shock and osmotic shock. They play a role in T cell differentiation and the cellular apoptosis pathway. As used herein the term JNK inhibitor refers to a compound which can inhibit the activity of any JNK.

Examples of JNK inhibitors include, but are not restricted to, SP600125, AS601245, JNK-IN-8, JNK-IN-7, JNK inhibitor VIII, IQ-3, DB07268, IQ-1S, AS602801. In some embodiments, the JNK inhibitor is SP600125.

The term antagonist of the aryl hydrocarbon receptor (AhR antagonist) as used herein refers to a compound which interferes or inhibits the activity of the aryl hydrocarbon receptor. The AhR is a member of the family of basic helix-loop-helix transcription factors that regulates gene expression as a sensor of xenobiotic chemicals such as aryl hydrocarbons and as a regulator of enzymes such as cytochrome P450s.

Examples of AhR antagonists include, but are not restricted to, SR1, PD98059, GNF351, BAY 2416964, CH-223191, PDM-11, BAY-218. In some embodiments, the AhR antagonist is SR1.

The term histone deacetylase inhibitor (HDAC inhibitor) as used herein refers to a compound which inhibits the activity of the enzyme histone deacetylase. There are four classifications of histone deacetylase; class I, class II, class III, and class IV. Based on their sequence homology and domain organisation, class II inhibitors can be further subdivided into class IIa and class IIb. As used herein the term histone deacetylase inhibitor refers to a compound which can inhibit the activity of any of the classes of histone deacetylase.

Examples of HDAC inhibitors include, but are not restricted to, Scriptaid, Vorinostat, Tacedinaline, RG2833, RGFP966, Trichostatin A, LMK235, Tubastatin A, Quisinostat, LBH589, PXD101, ITF2357, PCI-24781, FK228 MS-275, MGCD0103, Sodium Phenylbutyrate, Valproic acid, AN-9, Baceca, Savicol.

The term receptor tyrosine kinase (RTK) agonists as used herein refers to a compound which promotes the activity of the enzyme receptor tyrosine kinase. RTKs play an important role in cellular processes including growth, motility, differentiation and metabolism by catalysing phosphoryl transfer to tyrosine residues in protein substrates, using ATP as a phosphate donor. As such, dysregulation of RTK signalling leads to an assortment of human diseases including cancers. There are twenty classifications of RTKs.

Examples of RTK classifications include, but are not restricted to, the RET receptor family. Examples of RET agonists include, but are not restricted to GDNF, GFRα1, BT-13, Q525 and BT44.

An aspect of the invention includes the use of an aminothiol compound having the formula RNH(CₙH₂ₙ)NH(CₙH₂ₙ)SX, wherein R is hydrogen, an aryl, an acyl, or an alkyl group containing from 1 to 7 carbon atoms, each n has a value of from 2 to 6 and X is H or PO₃H₂; or a pharmaceutically acceptable salt thereof,

As used herein, “aryl” means a monocyclic, bicyclic, or tricyclic monovalent or divalent (as appropriate) aromatic radical, such as phenyl, biphenyl, naphthyl, anthracenyl, which can be optionally substituted with up to five substituents preferably selected from the group of C1-C6 alkyl, hydroxy, C1-C3 hydroxyalkyl, C1-C3 alkoxy, C1-C3 haloalkoxy, amino, C1-C3 mono alkylamino, C1-C3 bis alkylamino, C1-C3 acylamino, C1-C3 aminoalkyl, mono(C1-C3 alkyl)amino C1-C3 alkyl, bis(C1-C3 alkyl)amino C1-C3 alkyl, C1-C3-acylamino, C1-C3 alkyl sulfonylamino, halo, nitro, cyano, trifluoromethyl, carboxy, C1-C3 alkoxycarbonyl, aminocarbonyl, mono C1-C3 alkyl aminocarbonyl, bis C1-C3 alkyl aminocarbonyl,—SO3H, C1-C3 alkylsulfonyl, aminosulfonyl, mono C1-C3 alkyl aminosulfonyl and bis C1-C3-alkyl aminosulfonyl.

As used herein, “alkyl” means a C1-C7 alkyl group, which can be linear or branched. Preferably, it is a C1-C6 alkyl moiety. More preferably, it is a C1-C4 alkyl moiety. Examples include methyl, ethyl, n-propyl and t-butyl. It may be divalent, e.g. propylene.

As used herein, acyl is an alkyl group as defined above, which includes a carbonyl group (C═O).

Each of the alkyl and acyl groups may be optionally substituted with aryl, cycloalkyl (preferably C3-C10) or heteroaryl. They may also be substituted with halogen (e.g. F, Cl), NH₂, NO₂ or hydroxyl.

As used herein the term “umbilical cord blood” has its conventional use in the art; that is generally the blood that is left in the umbilical cord and placenta postpartum. Human cord blood is within the scope of the present invention and is obtained with written informed pre-consent and ethical approval.

As used herein the term “peripheral blood” has its conventional use in the art; that is generally blood which is circulating throughout the circulatory system. Human peripheral blood is within the scope of the present invention and is obtained with written informed pre-consent and ethical approval.

As used herein the term “bone marrow” has its conventional use in the art; that is, generally the gelatinous tissue present in bone cavities. The tissue comprises red bone marrow, a subset of bone marrow having populations of hematopoietic stem cells, progenitor cells and precursor cells. Human bone marrow is within the scope of the present invention and is obtained with written informed pre-consent and ethical approval.

As used herein the term “expanded cells” refers to cells which have been cultured ex vivo, under appropriate conditions, and undergone cell division to amplify the number of cells,

As used herein the term “cell expansion” refers to the amplification of the number of cells by the ex vivo culturing of cells under appropriate conditions, wherein the number of cells present at the end of culturing is greater than the number of cells present at the start of culturing.

Within the cells, wherein the cells may be part of the isolated population of cells, the cultured population of cells or the expanded cells, there are subtypes of cells. Examples of the cell subtypes are, but not restricted to; hematopoietic stem cells, hematopoietic progenitor cells and cells as defined by their phenotypic markers. Non-limiting examples of phenotypic markers are; Lin or CD38 or CD34 or CD133 or CD45RA or CD90 or CD49f or CD201, wherein the cells can also be defined by combinations of these phenotypic markers. As used herein the term “enriched” is used to refer to a set of cells which contains a high proportion of a specific subset/subtype of cell, wherein the set of cells may contain 2%, or 5%, or 10%, or 15%, or 20%, or 25%, or 30%, or 35%, or 40%, or 45%, or 50%, or 55%, or 60%, or 65%, or 70%, or 75%, or 80%, or 85%, or 90% of the specific subset/subtype of cell. Within the present invention the term “enriched” can be used to refer to a population of cells wherein the cells have undergone expansion and wherein a specific subtype of cells have increased in number proportionally more than other cells within the population. This enriched population of cells contains a significant proportion of a specific subtype of cells, wherein the significant proportion may be 2%, or 5%, or 10%, or 15%, or 20%, or 25%, or 30%, or 35%, or 40%, or 45%, or 50%, or 55%, or 60%, or 65%, or 70%, or 75%, or 80%, or 85%, or 90% of the total population.

As used herein the term “serum free tissue culture system” refers to culturing cells in a media which has not been supplemented with serum derived from an animal.

As used herein the term “feeder free tissue culture system” refers to a method of culturing cells without utilising a layer of connective tissue cells to support and provide metabolites to the growing cells.

As used herein the term “total cell expansion” refers to the increase in number of total nucleated cells.

As used herein the term “total culturing time” refers to the time in which steps ii), ili) and iv) are carried out. Wherein step ii) comprises culturing the isolated population of HSPC for at least 48 hours to form a first cultured population, step iii) comprises adding a histone deacetylase inhibitor (HDAC inhibitor), to form a second cultured population and step iv) comprises adding an aminothiol compound having the formula RNH(CₙH₂ₙ)NH(CₙH₂ₙ)SX, wherein R is hydrogen, an aryl, an acyl, or an alkyl group containing from 1 to 7 carbon atoms, each n has a value of from 2 to 6 and X is H or PO₃H₂; or a pharmaceutically acceptable salt thereof, to the cultured population of HSPC to form expanded cells. During the total culturing time, the cells are allowed to grow on an appropriate media such as basal media, the end of the total culturing time is signified by the cells being harvested, pooled or analysed.

As used herein, the term “pre-culturing step” refers to step ii), wherein step ii) comprises culturing the isolated population of HSPC for at least 48 hours to form a first cultured population and is separate from the isolation step of step i).

As used herein, the term “subject” refers to any animal (for example, a mammal), including, but not limited to, humans, non-human primates, canines, felines, rodents, and the like, which is to be the recipient of a therapy in accordance with the use of the present invention. Human subjects are envisaged in particular. “Patient” is used herein to refer to a human subject.

Aspects of the invention are defined above. The embodiments described below are applicable to all aspects of the invention.

The expanded population of cells produced by the method described herein may be enriched for HSC, and the expanded cells have been shown to have long term engraftment capabilities and the ability to repopulate mammalian bone marrow.

Kits of the invention are described herein. In a preferred embodiment the kit also contains apparatus and/or materials for obtaining an isolated population of HSPC. A person skilled in the art will know of suitable elements, apparatus and/or materials. Examples include magnetic bead isolation, MACS bead isolation columns, CliniMACS (Miltenyi) or FACS sorting.

In an embodiment of the present invention, the therapy or treatment is of a haematological disorder, immune disorder, metabolic disorder, or neurodegenerative disorder.

In some embodiments, the therapy or treatment comprises repopulating mammalian bone marrow.

In a preferred embodiment the therapy or treatment is for acute myelogeneous leukemia, acute lymphoblastic leukemia, chronic lymphocytic leukemia, chronic myelogeneous leukemia, non-Hodgkin lymphoma, severe aplastic anemia, severe combined immunodeficiency or sickle cell disease.

In one embodiment the therapy or treatment is gene therapy.

In one embodiment the method of treatment further comprises a step of administering the expanded cells to a subject.

In one embodiment step i) further comprises selecting for cells which are CD133+ and/or CD34+. In some embodiments, the isolated population comprises cells which are CD133+ and/or CD34+.

In some embodiments of the present invention, step i) comprises culturing the cells for about 12 hours to about 36 hours, preferably 24 hours. This is to allow them to recover from harvesting, transportation, cooling, freezing, thawing or any other such manipulations that may precede the pre-culturing step (step ii)). This culturing is sometimes referred to in the art as ‘recovery’ or ‘priming’. Preferably, the cells are recovered in StemSpan ACF media (Stem Cell Technologies) containing 100 ng/ml SCF, 100 ng/ml FLT3LG and 20 ng/ml TPO (all from Miltenyi Biotec) in 96 well round bottom suspension plates in a 37° C. humidified incubator with 5% CO₂.

If the isolated population of HSPC is obtained from a source that has been frozen it may be preferable to select for cells which are CD34+. If the isolated population of HSPC is obtained from a fresh source, then it may be preferable to select for cells which are CD133+. Suitable methods for selecting cells by cell surface markers are known in the art for example, by using Magnetic Activated Cell Sorting (MACs) or Fluorescent Activated Cell Sorting (FACS). Preferably, the isolated population comprises cells which are CD38− or CD34+ or CD133+ or CD45RA− or CD90+ or CD49f+, or any combination thereof.

In some embodiments of the present invention, the culturing process of step ii) is separate from the isolation (i.e., recovery or priming) process of step i).

In some embodiments of the present invention, step ii) comprises culturing the isolated population of HSPC for a period of time sufficient for the total number of viable nucleated cells to increase. In some embodiments, the total number of CD34+ will decrease.

In some embodiments of the present invention, step ii) comprises culturing the cells for about 48 hours to about 96 hours, preferably 72 hours,

In one embodiment the cells in the first cultured population are less pluripotent than the isolated population of HSPC, and the number of cells in the first cultured population have increased.

In one embodiment the cells in the first cultured population proliferate more than the cells in the second cultured population.

In one embodiment step iii) comprises adding the HDAC inhibitor at a time where the cells from the first cultured population have become less pluripotent that the isolated population of HSPC.

In some embodiments of the present invention, step iii) comprises culturing the cells for about 24 hours to about 72 hours, preferably 48 to 72 hours.

In some embodiments, the cells in the first cultured population of step ii) proliferate more than the cells in the second cultured population of step iii).

In some embodiments of the present invention, step iv) comprises culturing the cell for about 16 hours to about 24 hours, preferably 24 hours.

In some embodiments of the present invention, step ii) comprises culturing the cell in the presence of basal media.

In some embodiments of the present invention, step ii) comprises culturing the cells in the presence of compounds that are known to expand CD34+CD38− HSPCs. Suitable compounds are listed in the following documents, which are herein incorporated by reference: Sakurai Masatoshi et al: “Chemically defined cytokine-free expansion of human haematopoietic stem cells” (Nature 2023); and Saiyin et al: “Clinical Outcomes of Umbilical Cord Blood Transplantation Using Ex Vivo Expansion: A Systematic Review and Meta-Analysis of Controlled Studies” (Transplantation and Cellular Therapy 2022). In some embodiments, step (ii) comprises culturing the cells in the presence of nicotinamide, pyrimido-indole derivatives such as UM171 and UM729, p38 MAPK inhibitors, Notch ligands, Wnt agonists, a JNK inhibitor, a cytokine and/or an AhR antagonist.

In one embodiment of the present invention, step ii) comprises culturing the cell in the presence of a JNK inhibitor,

In some embodiments of the present invention, step ii) further comprises culturing the cell in the presence of an antagonist of the aryl hydrocarbon receptor,

In some embodiments of the present invention, step ii) further comprises culturing the cell in the presence of a cytokine, preferably an interleukin, more preferably interleukin 6 (IL-6).

In one embodiment of the present invention the JNK inhibitor is selected from SP600125, AS601245, JNK-IN-8, JNK-IN-7, JNK inhibitor VIII, IQ-3, DB07268, IQ-1S, AS602801. In a preferred embodiment, the JNK inhibitor is SP600125.

In one embodiment of the present invention the AhR antagonist is selected from SR1, PD98059, GNF351, BAY 2416964, CH-223191, PDM-11, BAY-218. In a preferred embodiment, the AhR antagonist is SR1.

In another embodiment of the present invention, step ii) further comprises culturing the cell in the presence of at least one, preferably at least two RET agonist(s).

In one embodiment of the present invention the at least one, preferably at least two RET agonist(s) is selected from GDNF, GFRα1, BT-13, Q525 and BT44 In a preferred embodiment, the at least one, preferably at least two RET agonist(s) is selected from GDNF and GFRα1. In some embodiments of the invention, two RET agonists are used in the culture of step ii).

In one embodiment of the present invention the HDAC inhibitor is selected from a broad-spectrum inhibitor, or a selective class I, class IIa, class IIb, class III or class IV inhibitor. Preferably, the HDAC inhibitor is selected from a broad-spectrum inhibitor, or a selective class I, class Iia, class III or class IV inhibitor. More preferably, the HDAC inhibitor is a broad-spectrum inhibitor, class I or class Ila inhibitor.

In a preferred embodiment, the HDAC inhibitor is selected from scriptaid, RG2833, RGFP966, LMK235, Tubastatin A, quisinostat, sodium phenylbutyrate. In some embodiments of the present invention, the HDAC inhibitor is a scriptaid or quisinostat. In a preferred embodiment the HDAC inhibitor is scriptaid, which has the structure;

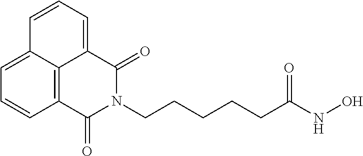 [SMILES: O=C(CCCCCN1C(=O)c2cccc3cccc(c23)C1=O)NO]

In a preferred embodiment, R is hydrogen.

In some embodiments, the aminothiol compound of the invention is amifostine:

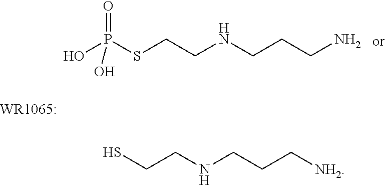 [SMILES: NCCCNCCSP(=O)(O)O ; NCCCNCCS]

In some embodiments of the present invention, the cells are washed between steps ii) and iii), preferably wherein the cell culture medium at stage iii) or after is substantially free of the JNK inhibitor and/or the antagonists of the aryl hydrocarbon.

In an embodiment of the present invention the JNK inhibitor is used at a concentration of 0.01 μM to 50 μM, preferably 0.1 μM to 10 μM, more preferably the JNK 10 inhibitor is used at a concentration at 0.2 μM.

In an embodiment of the present invention the antagonist aryl hydrocarbon receptor is used at a concentration of 0.01 μM to 50 μM, preferably 0.1 μM to 10 μM, more preferably the antagonist aryl hydrocarbon receptor is used at a concentration of 1 μM.

In an embodiment of the present invention the IL-6 is used at a concentration of 0.01 μg/ml to 50 μg/mlm preferably 0.05 μg/ml to 10 μg/ml, more preferably the antagonist aryl hydrocarbon receptor is used at a concentration of 0.1 μg/ml.

In an embodiment of the present invention the at least one, preferably at least two RET agonist(s) is used at a concentration of 100 ng/ml. Wherein more than one RET agonist is present, they are used in approximately equal amounts (1:1) at a concentration 20 of 100 ng/ml.

In an embodiment of the present invention the HDAC inhibitor is used at a concentration of between 0.01 μM to 50 μM, preferably between 0.1 μM to 10 μM, more preferably the HDAC inhibitor is used at a concentration of 0.3 μM.

In a preferred embodiment of the present invention the aminothiol compound e.g. WR1065, is used at a concentration of 50 μM to 500 μM, preferably 50 μM to 150 μM.

In some embodiments of the present invention, steps ii), iii) and iv) are performed over a total time sufficient for the isolated population of HSPC to form expanded cells.

In some embodiments of the present invention, steps ii), iii) and iv) are performed over two to ten days, preferably over three to eight days. In a preferred embodiment steps ii), iii) and iv) are performed over three to eight days. In a more preferred embodiment steps ii), iii) and iv) are performed over about five days.

In an embodiment of the present invention, steps iii) and iv) begin up to 72 hours before the end of the total culturing time (i.e. the end of step iv)).

In some embodiments of the present invention step iv) begin 16 to 24 hours before the end of the total culturing time.

Preferably, step iii) is performed 48 to 72 hours before the end of the total culturing time and step iv) is performed 24 hours before the end of the total culturing time.

Preferably, the cells are cultured in a serum free tissue culture system. In some embodiments of the present invention, cells are cultured in a feeder free tissue culture system. Whilst the culture system is serum and/or feeder free, various nutrients may be added to provide adequate growth and expansion conditions for cells. Examples of suitable media include, but are not limited to StemSpan ACF media (Stem Cell Technologies), StemPro34 serum-free medium (Invitrogen), Stemline II (Thermo Fisher), HPC Expansion Medium DXF (PromoCell), QBSF-60 (Quality Biological), StemMACS HSC expansion media XF (Miltenyi Biotec). In a preferred embodiment of the present invention the cells are cultured in StemSpan ACF media (Stem Cell Technologies). Suitable media may also contain various additives and components which may be chemical or biological components. These components may be incorporated into the suitable media singly or in combination and the skilled person will be able to choose suitable components as required. These components may also be incorporated during culture as required. Examples of components both biological and chemical include, but are not restricted to; amino acids, vitamins, cytokines, growth factors, hormones, antibiotics, fatty acids, saccharides, sodium, calcium, potassium, magnesium, phosphorus, agar, agarose, methylcellulose, collagen, insulin, transferrin, lactoferrin, cholesterol, ethanolamine, sodium pyruvate, 2-mercaptoethanol, polyethylene glycol, sodium selenite.

Various cytokines may be incorporated into the media and/or incorporated during culture, examples of suitable cytokines include, but are not restricted to; interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-18 (IL-18), interleukin-21 (IL-21), interferon-a (INF-a), interferon-β (INF-β), interferon-γ (INF-γ), granulocyte-macrophage colony stimulating factor (GM-CSF), stem cell factor (SCF), Wnt1, bone morphogenetic protein 7 (BMP7), angiopoietin-like 5 (ANGPTL5), insulin growth factor binding protein 2 (IGFBP2), erythropoietin (EPO), thrombopoietin (TPO), Fms-like tyrosine kinase 3-ligand (FLT3LG). In an embodiment of the present invention the media is supplemented with SCF, TPO and FLT3LG.

Various growth factors may be incorporated into the media and/or incorporated during culture. Examples of suitable growth factors include, but are not restricted to insulin-like growth factor (IGF), epidermal growth factor (EGF), human epidermal growth factor (hEGF), platelet-derived growth factor (PDGF), fibroblast growth factor 1 (FGF1), nerve growth factor (NGF), macrophage inflammatory protein 1-α (MIP-1α), leukaemia inhibitory factor (LIF).

In an embodiment of the present invention, the isolated population is cultured at a temperature between 32° C. to 39° C., preferably between 36° C. to 38° C. In an embodiment of the invention the cells are cultured in a humidified incubator with between about 1% to about 50% CO₂, preferably between about 1% to about 25% CO₂, more preferably between about 1% to about 10% CO₂. The present invention can be performed in a culture vessel suitable for animal cell culture. In one embodiment the present invention is performed in Nanex Hematopoietic Stem/Progenitor Cell (HSPC) Expansion Plates or TC treated Corning 24 well plates or suspension Greiner Bio 24 well plates. In a preferred embodiment, the present invention is performed in a conventional cell culture plate or a suitable closed system such as a cell culture bag (e.g VueLife®) or a stirred bioreactor.

In an embodiment of the present invention, the expanded cells are enriched for hematopoietic stem cells (HSC) and long-term hematopoietic stem cells (LT-HSC). In some embodiments the expanded cells are enriched for Lin− or CD38− or CD34+ or CD133+ or CD45RA− or CD90+, CD201+ or CD49f+ or CD201 or any combination thereof, preferably wherein the cells are enriched for CD34+, CD133+, more preferably wherein the expanded cells are enriched for CD38−, CD34+, CD133+, most preferably wherein the expanded cells are enriched for Lin−, CD38−, CD34+, CD133+, CD45RA−, CD90+, CD49f+ and preferably CD201.

In an embodiment of the present invention, the expanded cells are enriched for; Lin−, CD38−, CD34+, CD133+, CD45RA−, CD90+, CD201+ and CD49f+ or any combination thereof.

In a preferred embodiment of the present invention, the total cell expansion is between about 2-fold to about 50-fold, or from about 5-fold to about 50-fold, or from about 10-fold to about 20-fold. The total cell expansion is determined by measuring the number of total nucleated cells at the start of the culturing time and comparing to the number of total nucleated cells present at the end of the culturing time.

In a preferred embodiment, the expansion of Lin−, CD38−, CD34+, CD133+, CD45RA−, CD90+, CD201+ and/or CD49f+ cells is 200 to 2,000-fold, more preferably 400 to 1,000-fold, most preferably 800-fold. The expansion of Lin−, CD38−, CD34+, CD133+, CD45RA−, CD90+, CD201+ and/or CD49f+ cells is determined by measuring the number of Lin−, CD38−, CD34+, CD133+, CD45RA−, CD90+, CD201+ and/or CD49f+ cells present at the start of the culturing time and comparing it to the number of Lin−, CD38−, CD34+, CD133+, CD45RA, CD90+, CD201+ and/or CD49f+ cells present at the end of the culturing time. In an embodiment, the expansion of CD34+, CD45RA− and CD90+ cells is 50 to 800-fold, more preferably 400 to 600-fold, most preferably 500-fold. The expansion of CD38−, CD34+, CD45RA− and CD90+ cells is determined by measuring the number of CD38−, CD34+, CD45RA− and CD90+ cells present at the start of the culturing time and comparing it to the number of CD38−, CD34+, CD45RA− and CD90+ cells present at the end of the culturing time.

Suitable methods for determining cell expansion are known in the art and include, for example, multicolour flow cytometric analysis combined with total cell counting, use of absolute counting beads in combination with flow cytometric analysis, cell counts based on imaging analysis of a cell aliquot using a manual or automated hemocytometer (Viacell, Countess, Nucleocounter, Nexcelome).

Disclosed herein is an expanded population of cells, preferably HSC, wherein the expanded population is enriched for Lin−, CD38−, CD34+, CD45RA−, CD90+, CD201+ and/or CD49f+.

In one embodiment of the present invention, the isolated population of cells is obtained from umbilical cord blood or bone marrow or peripheral blood.

In a preferred embodiment, the isolated population of cells is obtained from umbilical cord blood.

In some embodiments, the cells are obtained from a mammal (for example mouse, rat, dog or human). A preferred embodiment is wherein the cells are obtained from a human.

The method to produce the expanded cells for use in therapy, the method of treatment and the use in the manufacture of a medicament as described above may comprise any of the additional features provided herein.

The cells expanded by the method presented herein can be used as a cell transplant. The cells expanded by the method presented herein may be used to repopulate mammalian bone marrow. Therefore, an embodiment of the present invention is an expanded population of cells for use in the treatment of a haematological disorder, immune disorder, metabolic disorder, or neurodegenerative disorder. Wherein the subject is administered an expanded population of cells which has been expanded according to the method described above. In a particular embodiment the expanded population of cells are for use in the treatment of a haematological disorder.

The expanded population of cells can be used as a graft for hematopoietic stem cell therapy as a substitute for conventional bone marrow, cord blood or peripheral blood transplantation. The transplantation of the expanded population of cells may be carried out in the same manner as conventional bone marrow, cord blood or peripheral blood transplantation. The graft may comprise the expanded population of cells along with any of the following components; a buffer solution, an antibiotic, a pharmaceutical compound.

Examples of disorders that may be treated using the expanded population of cells include acute myelogeneous leukemia, acute lymphoblastic leukemia, chronic lymphocytic leukemia, chronic myelogeneous leukemia, non-Hodgkin lymphoma, severe aplastic anemia, severe combined immunodeficiency, sickle cell disease, chronic granulomatosis, severe combined immunodeficiency syndrome, adenosine deaminase (ADA) deficiency, agammaglobulinemia, Wiskott-Aldrich syndrome, Chediak-Higashi syndrome, immunodeficiency syndrome such as acquired immunodeficiency syndrome (AIDS), C3 deficiency, congenital anemia such as thalassemia, hemolytic anemia due to enzyme deficiency and sicklemia, lysosomal storage disease such as Gaucher's disease and mucopolysaccharidosis, adrenoleukodystrophy, various kinds of cancers and tumors, especially blood cancers such as acute or chronic leukemia, Fanconi syndrome, aplastic anemia, gramulocytopenia, lymphopenia, thrombocytopenia, idiopathic thrombocytopeni purpura, thrombotic thrombocytopenia purpura, Kasabach-Merritt syndrome, malignant lymphoma, Hodgkin's disease, multiple myeloma, chronic hepatopathy, renal failure, massive blood transfusion of bank blood or during operation, hepatitis B, hepatitis C, severe infections, systemic lupus erythematodes, articular rheumatism, xerodermosteosis, systemic sclerosis, polymyositis, dermatomyositis, mixed connective tissue disease, polyarteritis nodosa, Hashimoto's disease, Basedow's disease, myasthenia gravis, insulin dependent diabetes mellitus, autoimmune hemolytic anemia, snake bite, hemolytic uremic syndrome, hypersplenism, bleeding, Bernard-Soulier syndrome, Glanzmann's thrombasthenia, uremia, myelodysplasia syndrome, polycythemia rubra vera, erythremia, essential thrombocythemia, myeloproliferative disease, traumatic spinal cord injury, nerve injury, neurotmesis, skeletal muscle injury, scarring, diabetes mellitus, cerebral infarction, myocardial infarction, and obstructive arteriosclerosis.

The expanded population of cells may be administered through the following administration routes; subcutaneous, intraparietal, intramuscular, intravenous, intratumor, intraocular, intraretinal, intravitreal, or intracranial.

The expanded population of cells may be combined with a pharmaceutically acceptable excipient, diluent, or carrier in order to improve and enhance administration, stability, uniformity, bioavailability, or any combination thereof. In certain embodiments, the extracellular vesicles or cells of the current disclosure are administered suspended in a sterile solution. In certain embodiments, the solution comprises 0.9% NaCl. In certain embodiments, the solution further comprises one or more of: buffers, for example, acetate, citrate, histidine, succinate, phosphate, bicarbonate, or hydroxymethylaminomethane (Tris); surfactants, for example, polysorbate 80 (Tween 80), polysorbate 20 (Tween 20), or poloxamer 188; polyol/disaccharide/polysaccharides, for example, glucose, dextrose, mannose, mannitol, sorbitol, sucrose, trehalose, or dextran 40; amino acids, for example, glycine or arginine; antioxidants, for example, ascorbic acid or methionine; and chelating agents, for example, EGTA or EGTA.

The expanded population of cells produced by the present method may be used in gene therapy. In order to introduce a therapeutic gene to a patient, the gene of interest should be transfected into the HSC of the isolated population. The therapeutic gene can be introduced using viral or non-viral methods. Suitable viral vectors include retrovirus, adenovirus, adeno-associated virus and herpes-simplex virus. The cells comprising the gene of interest can be expanded according to the present method before being introduced to the patient.

The following examples illustrate the invention.

### EXAMPLES

### Protocol for Cell Expansion

### INTRODUCTION

Umbilical Cord Blood (UCB) is a valuable source of HSC for patients that require allogeneic transplantation, especially those from ethnic minorities for whom HLA matched donors are hard to identify. Despite allowing for less stringent HLA matching and its ability to confer superior overall and relapse-free survival, UCB transplantation potential is constrained by the limited number of HSCs in each UCB unit, variability in the expansion between UCB units as well as delayed hematopoietic recovery compared to GCSF-mobilized peripheral blood or BM transplant.

There are different approaches for expanding the UCB CD34+ cells without using their regeneration potential.

Currently there are two companies with an expanded UCB offer: ExCellthera, and Gamida Cell (the latter most advanced being in Phase 3 Clinical stage).

### Materials and Methods

### Cells

Cryopreserved human Umbilical cord blood (UCB) cells enriched for CD34+ cells were obtained (after sorting with magnetic beads). All experiments were performed starting from thawed frozen cell stock.

### Materials

1. Base media: StemSpan (Stem Cell Technologies)
2. Growth factors:

| Growth factors | Final concentration |
|---|---|
| SCF | 100 ng/ml |
| FLT3L | 100 ng/ml |
| TPO | 20 ng/ml |
| IL-6* | 100 ng/ml |

*IL-6 is not used as part of basal media

3. Chemical compounds:

| Compound | Final concentration | |
|---|---|---|
| Scriptaid | 300 | nM |
| WR-1065 | 100 | uM |
| SR1 | 1 | uM |
| SP600125 | 200 | nM |

| | |
|---|---|
| GDNF/GFRα1 | 100 ng/ml (1:1) |

### Protocol for Cell Expansion

Day 0: CD34-positive UCB cells were thawed and pooled in the base media StemSpan ACF media (Stem Cell Technologies) containing 100 ng/mL SCF, 100 ng/ml FLT-3L and 20 ng/mL TPO (all from Miltenyi Biotec). After counting, cells were plated in 96-well round bottom suspension plates at 20,000 cells/well in the above media. Cells were allowed to recover overnight in these conditions in a 37° C. humidified incubator with 5% CO₂.
Day 1: The recovered CD34-positive cells were harvested from the plates counted, and seeded into 24-well Nanex plates (Compass Biomedical) or standard tissue culture treated plates at 20,000 cells/well in 0.5 mL basal media containing the following components:

| Protocol | Basal media |
|---|---|
| Control | Basal media alone |
| PTC13303 | Basal media |
| (Comparative) | Scriptaid 300 nM |
| PTC13325.1 | Basal media |
| | SR1 1 uM |
| | SP600125 200 nM |
| | IL-6 100 ng/ml |
| PTC13325.2 | Basal media |
| | SR1 1 uM |
| | SP600125 200 nM |
| | IL-6 100 ng/ml |
| RET agonists | Basal media |
| | RET agonists (rhGDNF and rhGFRα1) |
| | 100 ng/ml (1:1) |
| PTC13325.3 | Basal media |
| | RET agonists (rhGDNF and rhGFRα1) |
| | 100 ng/ml (1:1) |

PCT13303 was disclosed in WO2020/084310 and WO2020/084310 and is included herein for comparative purposes. PTC13303 lacks step (ii) of the invention, i.e., a pre-culturing step before adding the HDAC and aminothiol.
Day 4: Cells from each well were collected separately, centrifuged and placed in the fresh media to follow the corresponding protocols:

| Protocol | Basal media |
|---|---|
| Control | Basal media |
| PTC13303 | Basal media |
| (Comparative) | Scriptaid 300 nM |
| PTC13325.1 | Basal media |
| | Scriptaid 300 nM |
| PTC13325.2 | Basal media |
| | Scriptaid 300 nM |
| | SR1 1 uM |
| | SP600125 200 nM |
| | IL-6 100 ng/ml |
| RET agonists | Basal media |
| | RET agonists (rhGDNF and rhGFRα1) |
| | 100 ng/ml (1:1) |
| PTC13325.3 | Basal media |
| | Scriptaid 300 nM |
| | RET agonists (rhGDNF and rh GFRα1) |
| | 100 ng/ml (1:1) |

Day 6: To protocols PTC13303 (comparative), PTC13325.1, PTC13325.2 and PTC13325.3, WR-1065 was added directly to the wells containing cell culture to the final concentration of 100 μM for a maximum of 24 hours. Cells from the control group were left untreated.
Day 7: Cells were collected from individual wells, centrifugated, and re-suspended in media/buffers as necessary for the following procedures:
   1. Flow cytometry
   2. Colony assays
   3. Animal transplantation
   4. RNA-sequencing

### Flow Cytometry of Expanded Cells

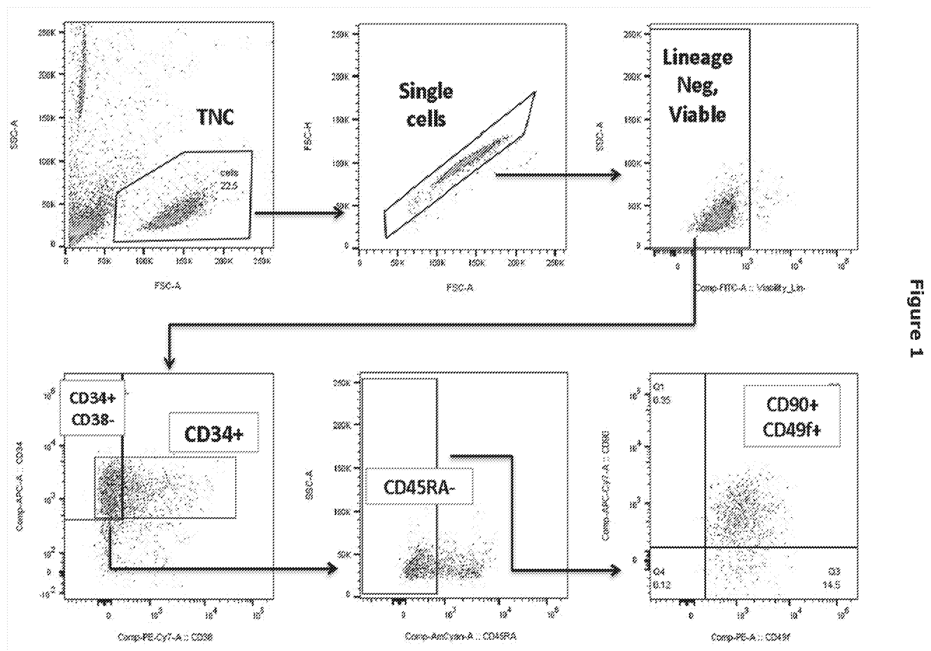

Cells were harvested, washed and stained in 3% FBS in PBS using a panel of pre-conjugated antibodies as shown in Table 1. Cells were incubated with antibodies on ice for 30 min, then washed twice in 3% FBS in PBS and resuspended in 250 uL of above buffer before being analysed using a BD Canto II flow cytometer (FIG. 1).

TABLE 1
Reagents for flow cytometry

| Antibodies | Fluorophores |
|---|---|
| Lin- | FITC |
| CD34 | APC |
| CD38 | PE-Vio770 |
| CD45RA | Viogreen |
| CD90 | APC-Vio770 |
| CD49f* | BV421 |
| CD201 (EPCR)* | PE |
| Viability dye: | |
| Fixable Viability Dye eFluor 540 | |

*CD49f and CD201 markers were not used together. The exact panel of antibodies used is mentioned in figure legends.

### Human Hematopoietic Colony Forming Assay in Semi-Solid Media

CFU assays were performed using the MethoCult Classic kit (Stem CellTechnologies) under manufacturer's instructions and the reagent shown in Table 2. Briefly, an aliquot of cells (either known number or volume) was mixed with 3 mL of MethoCult media and plated using a blunt needle and syringed into one well of a 6-well suspension plate. Plates were incubated for 2 weeks at 37° C. in a humidified incubator with 5% CO₂ without media change. After 2 weeks, colonies were photographed under a dissecting microscope and counted. The proportion of colonies of different kinds was then scored and the proportion/sample was calculated,

TABLE 2
Reagents for colony assay
Semi-solid media

MethoCult H4434 Classic with
recombinant cytokines

### Animal Transplantation

NSG mice were gamma irradiated 24 hrs before transplantation. Total cell numbers of in vitro expanded cells from each well were transplanted per each mouse intravenously. Cell numbers after expansion were deemed equivalent to the initial number of 20,000 of pre-expanded cells per well.

Mouse Bone Marrow aspirations for the analysis of human cell engraftment (CD45 content by flow cytometry) were carried out at weeks 10 and 16. Mice were sacrificed at 18 weeks post transplantation, Bone Marrow collected, and the content of human cells was analysed by flow cytometry using following markers: CD34, CD45RA, CD90, CD49f to determine percent of human leukocytes (CD45+) and human LT-HSCs (CD34+/CD45RA−/CD90+/CD49f+) in the mouse BM.

### Single Cell RNA Sequencing

The expanded human CD34 cells were sorted by flow cytometry for the LT-HSC cells for the following markers: CD34+/CD45RA−. This enriched population was processed for single cell RNA sequencing using standard procedure,

### Donor Deconvolution

FASTQ files were aligned to the human genome hg38 release 108 using STAR v.2.7.10a and the following parameters: --soloType CB_UMI_Simple--soloUMIlen 12--outSAMtype BAM SortedByCoordinate--runRNGseed 1-outSAMattributes NH HI AS nM CB UB GX GN. Aligned BAM files were indexed using samtools v. 1.16.1 with the samtools index function.

A custom VCF file was downloaded as indicated in the souporce//Github repository (https://github.com/wheaton5/souporcell), containing SNPs from the 1K Genomes project filtered for MAF>0.02.

The VCF file was indexed using bcftools from samtools v.1.12, with the bcftools index and bcftools tabix functions.

For every protocol, cells were genotyped using cellsnp-lite v 1.2.2 using the custom VCF as reference, barcodes filtered by CellRanger as input barcodes, and the parameters --minMAF 0.1--minCOUNT 20.

Then, donors were deconvoluted using the cellsnp-lite input with vireo v 0.2.3, using the same VCF file as reference and N=3 donors.

Genotyped donors were matched across protocols by importing the protocol VCF files in R v. 4.2.1 and calculating the Spearman correlation for the Allele Depth (AD) for each variant in each donor and protocol. 3 clusters emerged clearly and were used to assign matching donor identities across protocols.

### Filtering, Dimensionality Reduction and Integration

For quantification, FASTQ files were aligned and quantified using CellRanger v3 with default parameters. The resulting filtered count matrix was read into the R using the DropletUtils R/Bioconductor package v 1.18.1.

Donor identities from the deconvolution step were added to each dataset, and cells marked as “unassigned”, which are doublets and/or cells that failed genotyping, were discarded.

All count matrices were pooled together retaining sample identity. For quality control, cells were retained according to the following criteria:
1. Total read count within 3 Median Absolute Deviations (MADs) of the per-protocol median (filtering both low and high counts).
2. Percentage of mitochondrial transcripts within 3 MADs of the per-control median (high filter).
3. Percentage of MALAT1 expression within 3 MADs.

All filtering was done using the isOutlier function from the scater R/Bioconductor package v 1.26.1. This filtering resulted in a total of 16481 cells.

Next, counts were normalized using the computeSumFactors pooling method from the scran R/Bioconductor package v 1.26.1, including a pre-clustering step clustering within a combined factor of protocol and donor.

Highly variable genes were identified by fitting a non-linear trend to the mean-variance relationship (blocking by protocol) and retaining the top 2000 genes, ordered by their residuals from the fit, as implemented in modelGeneVar from scran.

For linear dimensionality reduction, log-normalized highly variable genes were used as input to the runPCA function from scran, resulting in 50 principal components. UMAP non-linear dimensionality reduction was run on the first 20 dimensions of the PCA embeddings, using the umap function from the uwot R package v 0.1.14 using the rounded square root of the number of cells as n_neighbors and setting min_dist=0.7.

For integration, the first 20 principal components of the PCA were used as input to the fastMNN function from the batchelor R/Bioconductor package v 1.18.1, using the combined donor and protocol factor as batch. Upon integration, UMAP reduction was performed again using the same parameters. UMAP was used exclusively for visualization and to quickly assess integration efficiency by comparing plots before and after integration.

### Multi-Resolution Clustering and Cell Type Annotation

First, a Shared Nearest-Neighbor (SNN) graph was constructed in the integrated space (first 20 dimensions) setting the number of shared neighbors k=10 and weighing edges between any two nodes by the Jaccard coefficient of their shared neighbors. This was carried out using the makeSNNgraph function from the bluster R/Bioconductor package v 1.8.0. Then, the Leiden graph-based community detection algorithm was applied to the SNN using 3 different values for the resolution parameter (0.2, 0.5, 0.8,1) using the cluster_leiden function from the igraph R package v 1.3.5, using 30 iterations.

To assess the most likely clustering resolution, three methods were used:
1. Constructing a clustering tree using the clustree R package v 0.5.0 and choosing a resolution that did not result in cross-overs between resolutions
2. Calculating the pairwise modularity ratio between clusters using the pairwiseModularity function from bluster
3. Calculating the approximate silhouette width by calculating the distance of each cell to each cluster centroid, as implemented in the approximateSilhouette function from bluster and choosing a resolution that maximizes silhouette widths overall

The combination of these criteria resulted in the choice of a resolution of 0.46 for clustering.

### Cluster Marker Identification

A Wilcoxon rank sum test was used to determine “cluster markers” as genes that are specifically highly expressed in one cluster compared to the rest of the data, using the wilcoxauc function from the presto R package (v. 1.0.0). Genes were ordered within each cluster by the difference between the percentage of cells they were expressed in and the percentage of cells in the rest of the clusters, and the union of the top 3 genes for each cluster was used to plot the dot plot.

### Cell Type Signature Analysis

The U-statistic was calculated using the UCell R/Bioconductor package v 2.6.2 using CD34+, PROCR+ and CD38− as input, with no other changes to default parameters.

### Differential Abundance Analysis

The R/Bioconductor package MiloR (v. 1.10.0) was used to assess protocol-associated differences in local abundance using donors as replicates. Neighbourhoods were built using k=30 on the fastMNN corrected space, taking in consideration only cells expanded (i.e. excluding cells from the uncultured samples). Then, differential abundance was tested using the testNhoods function using a design that included both protocol and sex as covariates, and spatial FDR correction was performed automatically by the function.

### Data Visualization

All figures for the single cell analysis results were plotted using the ggplot2 R package (v. 3.4.4).

### Example 1 In Vitro Expansion of Human UCB CD34-Enriched Cells by PTC13303, PTC13325.1 and PTC13325.2 Increases Numbers of Long-Term HSCs

The cells from four independent donors were expanded by the protocols: Ctrl-Control, PTC13303, PTC13325.1 and PTC13325.2. Cells were collected on Day 7 after expansion. Cell numbers as a result of expansion were quantified by flow cytometry in comparison to the counting beads used as an internal control. The cell populations were assessed by flow cytometry using the antibody panel shown in Table 1.

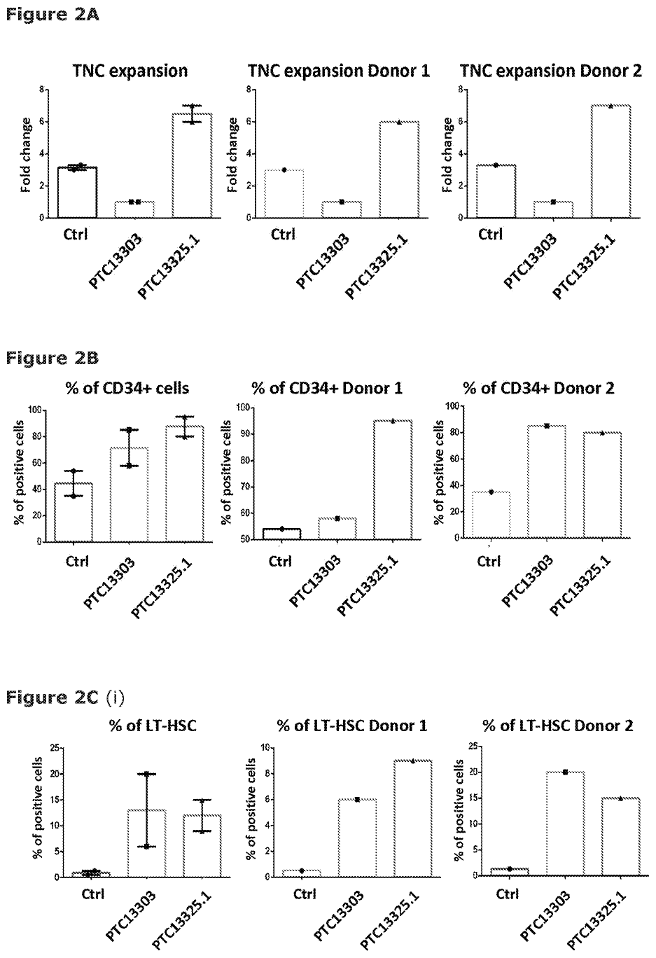

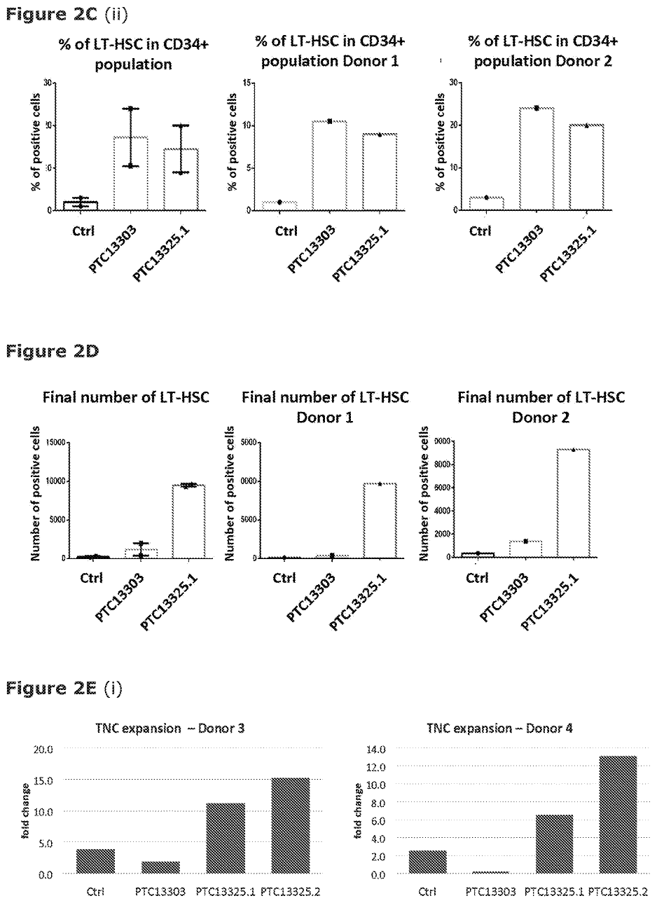

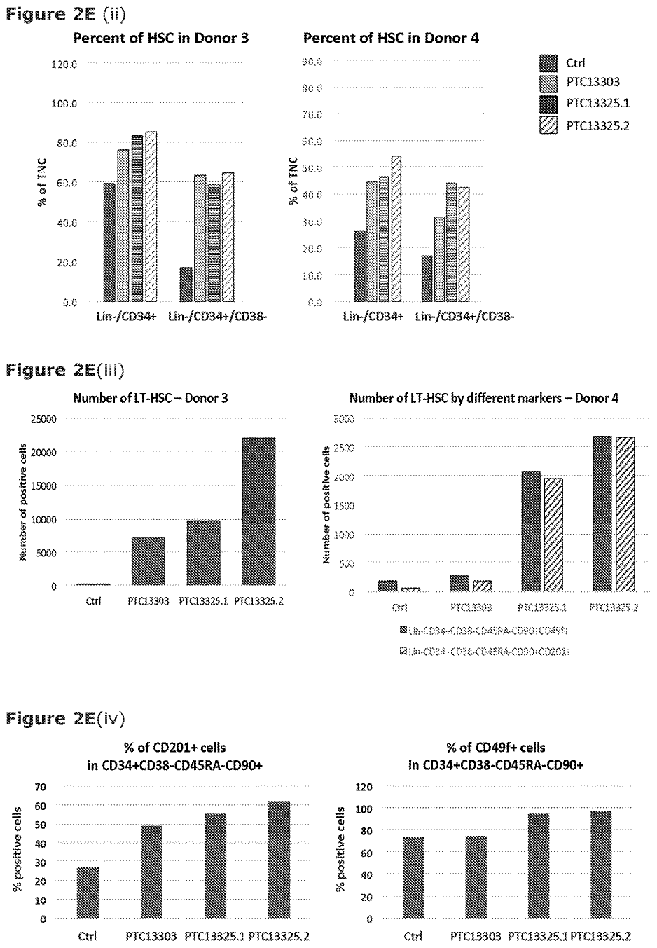

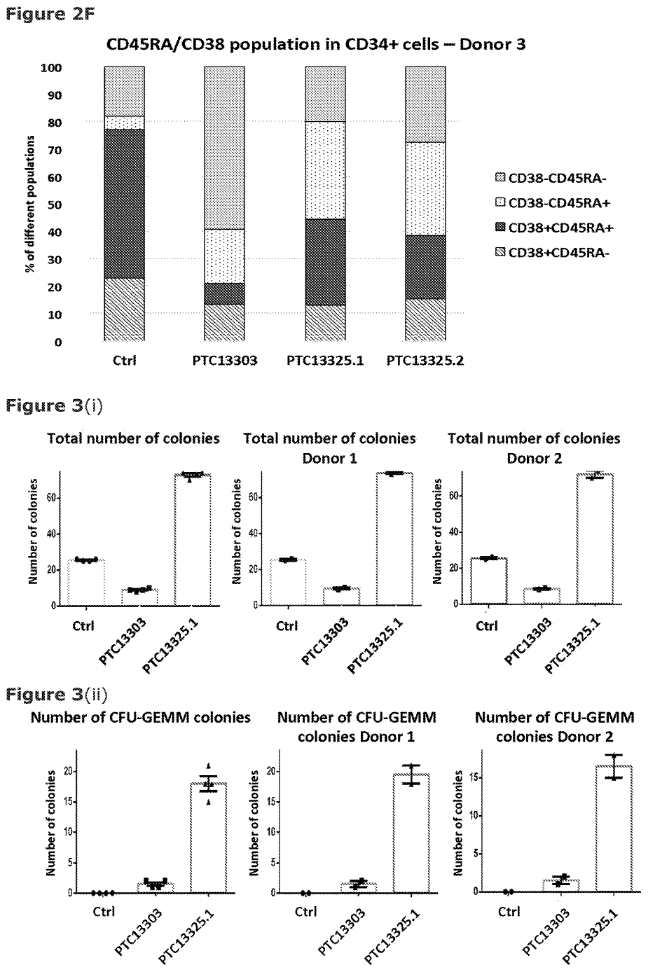

As shown in FIG. 2A, the increase in Total Nucleated Cell (TNC) number compared to the input cell number was higher, when cells were treated by PTC13325.1 as compared to the control treatment or treatment by PTC13303. Specifically, treatment with PTC13303 decreased TNC numbers compared to the control treatment.

The percent of CD34+ cells was increased after treatment with both PTC13303 and PTC13325.1 compared to that in Control treatment (FIG. 2B). Yet, as shown on graphs for two independent donors, the magnitude of this effect might be donor dependent. Similarly, treatments by both PTC13303 and PTC13325.1 increased the percent of long-term HSC (LT-HSC, defined as CD34+/CD38−/CD45RA−/CD90+/CD49f+, Table 1, FIG. 1) when estimated as a percent of LT-HSC within the TNC population (FIG. 2C (i)) or within the population of CD34-positive cells (FIG. 2C (ii)). The magnitude of this effect might be donor dependent as well.

However, a very clear donor-independent effect is observed when the total number of long-term HSCs was calculated using TNC numbers and a percent of LT-HSC within TNC population. As shown in FIG. 2D, the treatment with PTC13325.1 dramatically increases numbers of LT-HSCs compared to both control and PTC13303 treatments.

PTC13325.1 and PTC13325.2 were compared in vitro using UCB from 2 additional donors. Cells were processed as in the previous experiments using Donors 1 and 2. By the end of expansion, cells were assessed by flow cytometry to evaluate their numbers and phenotype using two different panels of markers: CD34+/CD38−/CD45RA−/CD90+/CD49f+ and CD34+/CD38−/CD45RA−/CD90+/CD201 (EPCR)+. The results are shown in FIG. 2E.

FIG. 2E (i) shows the fold change in the total number of TNC compared to that of the pre-expanded cells for two independent UCB donors (Donor 3 and Donor 4) after expansion using protocols: Ctrl, PTC13303 (comparative), PTC13325.1 and PTC13325.2. FIG. 2E (ii) shows the estimated percent of Lin−/CD34+ and Lin−/CD34+/CD45RA− cells after treatment with protocols: Ctrl, PTC13303, PTC13323.1 and PTC13325.2 for Donor 3 and Donor 4. FIG. 2E (iii) shows the total number of LT-HSCs (defined either by markers CD34+/CD38−/CD45RA−/CD90+/CD49f+ or by markers CD34+/CD38−/CD45RA−/CD90+/CD201+ (EPCR)) calculated using TNC numbers and percent of LT-HSC within TNC populations for Donor 3 and Donor 4. FIG. 2E (iv) shows percent of CD201+ or CD49f+ cells in the population of CD34+/CD38−/CD45RA−/CD90+ cells (Donor 4) after treatment with protocols: Ctrl, PTC13303, PTC13323.1 and PTC13325.2.

### Example 2 Hematopoietic Colony Formation Assay in Semi-Solid Media for In Vitro Expanded Cells

The standard in vitro test that provides an estimation of the HSC potency to engraft is a hematopoietic colony-forming assay in semi-solid media. This assay evaluates the number of colony-forming units (CFU) and their phenotype (erythroid, myeloid, lymphoid, or mixed (GEMM) colonies) that corresponds to the number and type of hematopoietic progenitor cells in the sample. The GEMM colonies represent the most primitive type of non-committed hematopoietic progenitors that are capable to produce both erythroid, myeloid and lymphoid blood cells.

1000 cells, collected after 7 days of expansion of CD34-enriched UCB cells from two independent donors using Ctrl, PTC13303 and PTC13325.1, were mixed with MethoCult semi-solid media and plated in duplicates for human hematopoietic colony formation for the period of 2 weeks. Number and type of hematopoietic colonies (colony forming units, CFU) were scored under the microscope. As it is shown in FIG. 3, cells treated by PTC13325.1 produce substantially higher numbers of hematopoietic colonies in general (FIG. 3(i)) and, specifically, colonies of the mixed GEMM phenotype (FIG. 3(ii)). Interestingly, cells expanded by PTC13303 had a higher potency for producing GEMM colonies compared to the cells expanded by the Ctrl (FIG. 3(ii)), but the total number of hematopoietic colonies is lower (FIG. 3(i)). This suggests that PTC13325.1 expands both progenitor and primitive stem cell compartments, while PTC13303 (comparative) favours only the expansion of primitive stem cells at the expense of progenitor cell expansion. This is important for engraftment and for reconstituting the immune system.

### Example 3 Engraftment of Expanded Human CD34-Positive Cells in NSG Mice

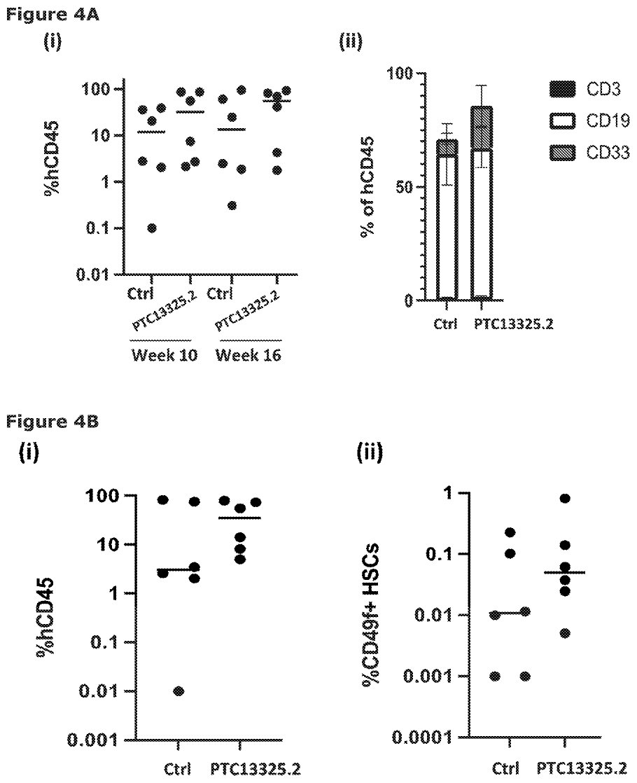

One of the most reliable and widely accepted criteria for the evaluation of potency of hematopoietic stem and progenitor cells is the ability of these cells to reconstitute the hematopoietic system of transplanted animal recipients. The NSG mice do not develop a fully functional immune system and, upon sub-lethal irradiation, are used as model recipients for hematopoietic xenografts. To test the reconstitution potential of in vitro expanded cells, NSG mice were transplanted with human UCB CD34-pos cells pooled from four donor units and expanded by Ctrl and PTC13325.2. The percent of hematopoietic reconstitution by human cells was estimated using human specific CD45 antibodies (FIG. 4).

Human/mouse chimerism was initially assessed using Bone Marrow aspirations (BMA) from the living animals at weeks 10 and 16 after transplantation (FIG. 4A). The percent of human cells was significantly higher in the BM of mice transplanted with cells expanded by PTC13325.2 compared to the Control protocol (FIG. 4A(i)), showing the increased development for both lymphoid and myeloid cells (FIG. 4A(ii)). At 18 weeks post transplantation, animals were sacrificed, and the presence of human cells was evaluated in the BM by flow cytometry using the panel of markers shown in Table 1. The percent of engrafted human cells was significantly higher in mice transplanted by cells expanded by PTC13325.2 reaching almost 100% in some animals (FIG. 4B (i)). Moreover, the percent of human LT-HSC defined by CD34+/CD38−/CD45RA−/CD90+/CD49f+ (CD49f+HSCs) markers was increased in the same animals compared to mice transplanted by cells expanded by the Control protocol (FIG. 4B (ii)).

### Example 4 Single Cell Transcriptomics Analysis

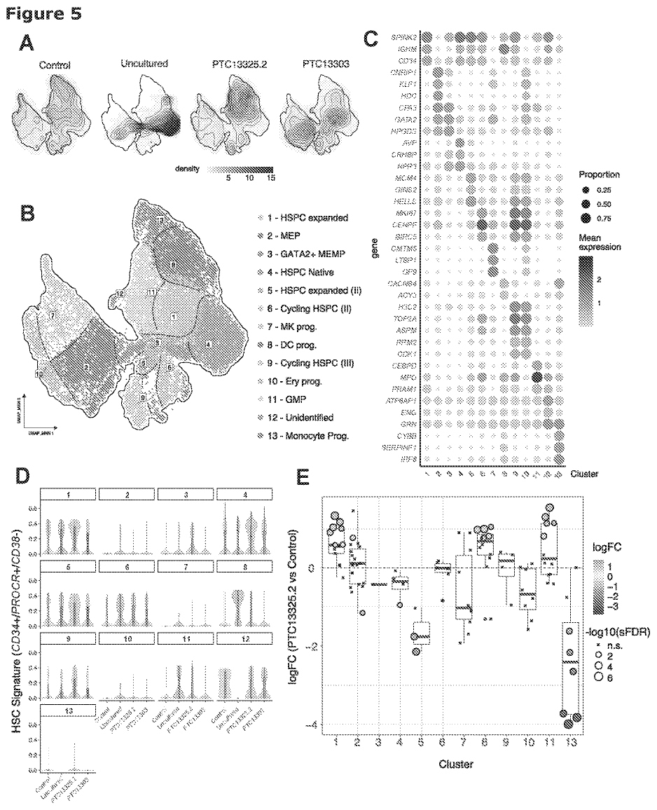

Analysis was done by single cell RNA sequencing using cells that were either freshly isolated from cord blood (Uncultured) or expanded for 7 days in either the control condition, protocol PTC13325.2, or protocol PTC13303. Three UCB donors were pooled for each condition, with control and protocols PTC13325.2 and PTC13303 sharing the same 3 donors. To assign donor identities to each cell Bayesian variant-based donor deconvolution was performed, using a 10,000 genome SNP catalogue as reference for common variants and identifying 3 donors per sample. After assigning each cell passing minimal QC filtering to each donor, an additional round of QC-based filtering was performed (see Methods) which resulted in 3721 cells (Uncultured), 7039 cells (Control), 4975 cells (protocol PTC13303), and 4558 cells (protocol PTC13325.2). Cells were integrated into a shared reduced dimensional space removing differences associated to sex and protocol and projected in 2 dimensions using UMAP (Uniform Manifold Approximation and Projection) (FIG. 5A). Multi-resolution Leiden clustering was performed and 12 clusters were identified at a resolution of 0.5 (FIG. 5B), which were annotated according to the expression of typical markers (FIG. 5C) such as CD34, SPINK2 (HSPCs), MKI67, TOP2A (cycling cells), CLU, ITGA2B (megakaryocytic progenitors), KLF1 (erythroid progenitors), GATA2, CPA3 (early megakaryocytic/erythroid/mast cell progenitors), CEBPD, MPO (Granulocyte-Monocyte Progenitors, GMP), IRF8, SERPINF1 (monocyte progenitors).

To better measure the increase in stem cell content caused by culturing in different media, a signature score was derived for each single cell in each cluster, and compared across protocols on a per-cluster basis (FIG. 5D), showing that both PTC13303 and PTC13325.2 increase the amount of cells with a higher HSC signature in clusters harbouring HSCPs and cycling HSPCs (clusters 4, 5, 6 and 9) compared to the Control expansion media, with PTC13325.2 showing a bigger increase than PTC13303.

The choice of cluster numbers in an in vitro expansion of a stem cell population (or mixture of progenitors) is indeed rather arbitrary, as cells exist in a continuum rather than a defined set of subpopulations. To better characterise this continuum the data was divided in partially overlapping neighbourhoods, which offered a more granular resolution while retaining even sampling of the transcriptional space (see Methods). Since there were three matched replicates between control and protocol PTC13325.2 conditions, the neighbourhood-level differential abundance was estimated using miloR (FIG. 5E), and it could be observed how a small but statistically significant set of neighbourhoods was positively over-represented in protocol PTC13325.2 containing cells corresponding to the expanded HSPC/MPP cluster (cluster 1, FIG. 5E). Conversely, the Control basal protocol seemed to be more biased towards other progenitor compartments, such as the monocytic progenitors (cluster 13).

Taken together, these findings all point to a preferential expansion of the long-term HSC compartment driven by protocol PTC13325.2 compared to the Control one, in agreement with the observations from the phenotyping and in vivo experiments.

### Example 5 Discovery of Protocols for the Expansion of Hematopoietic Progenitor and Stem Cells Using CombiCult Screening Platform and Ex Vivo Expanded UCB Cultures

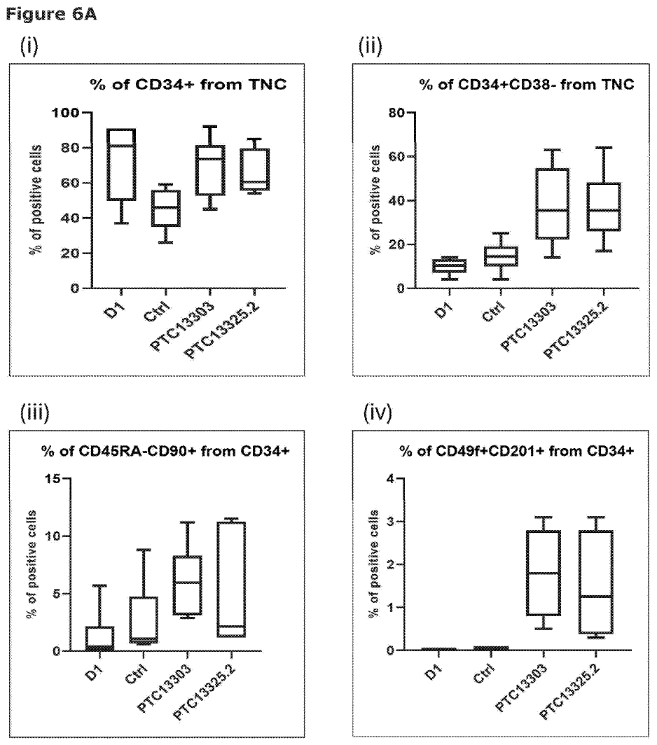

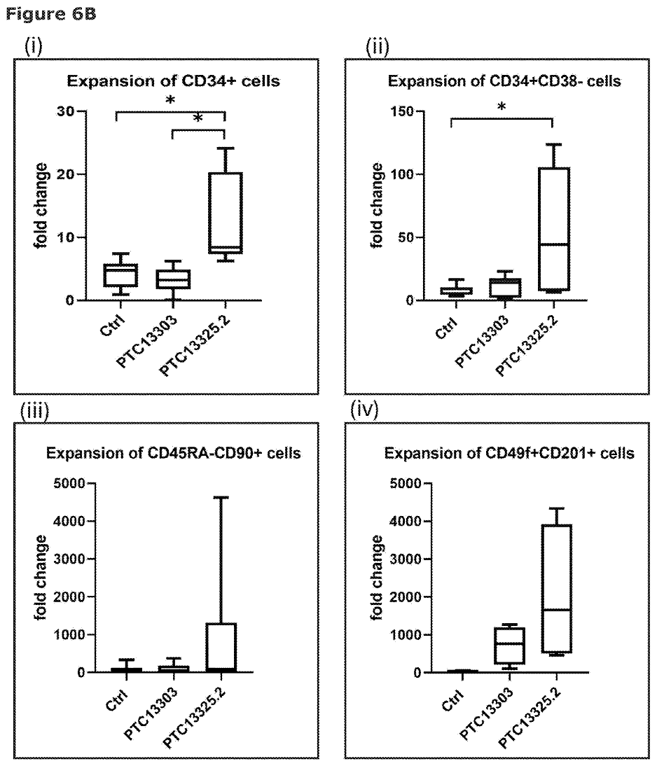

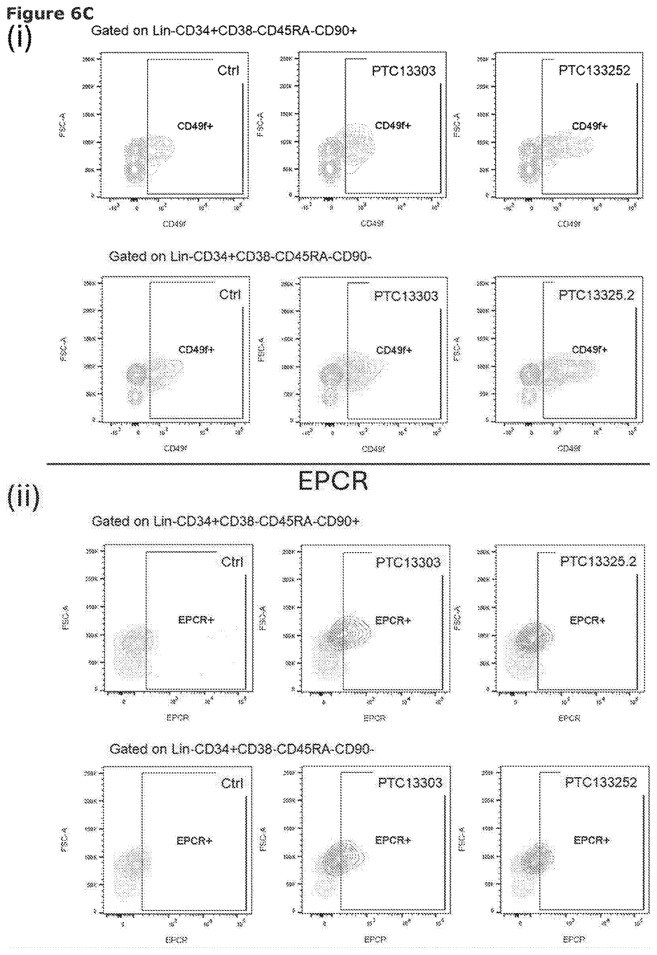

The ex vivo culturing of UCB cells with cytokine cocktails is known to promote cell divisions. These proliferative steps are associated with rapid loss of multipotent characteristics of HSCs and accumulation of advanced progenitors and differentiated cells, which are less capable of engraftment. The final product of such ex vivo culture, albeit having higher total cell numbers, has no long-term benefits in clinical applications. Following the 1-week of ex vivo culture in a cytokine cocktail with the addition of chemical combinations for protocols PTC13303 and PTC13325.2, the reproducible enrichment of CD34+ was observed (FIG. 6A(i)), and, most importantly, more primitive CD34+CD38− compartments were observed (FIG. 6A(ii)). Both protocols were able to significantly enrich for populations of engraftable CD34+CD38−CD45RA-CD90+ cells (FIG. 6A (iii)), which expressed markers associated with phenotype of Long-Term repopulating cells (LT-HSC, CD49f+EPCR+) (FIG. 6A(iv), FIG. 6C—FACS plots). Interestingly, the up-regulation of both CD49f and EPCR markers was observed in both CD90+ and CD90− cell populations compared to Day 1 pre-expanded cells and cells expanded in cytokines alone (Control), suggesting that media components in protocols PTC13303 and PTC13325.2 positively regulated the expression of those markers (FIG. 6C).

The increased production of phenotypically engraftable ex vivo treated cells per same amount of starting material in cells expanded by protocol PTC13325.2 compared to protocol PTC13303 was observed, although the magnitude of observed effect was donor dependent (FIG. 6B).

This Example shows that ex vivo expanded UCB cultures were enriched for HSCs.

### Example 6 Expansion of Primitive HSCs

Mechanistically, both protocols PTC13303 and PTC13325.2 exploit the use of HDAC inhibitor (Scriptaid) in combination with additional small molecules, which have relatively studied functions in the context of stem cells. StemRegenin 1 (SR1) is an AHR inhibitor positively affecting HSC expansion, and JNK inhibitor (SP600125) and WR playing various roles in stress response, cell cycle and differentiation. Following the comparison of both protocols in ex vivo cultures, it was proposed that the similarity in expansion of the primitive cell compartment could be attributed to the use of HDAC inhibitors (shared by both protocols, FIG. 6A), while the higher numbers of more primitive cells obtained by protocol PTC13325.2 benefitted from the addition of the expansion step during first 3 days in culture (FIG. 6B).

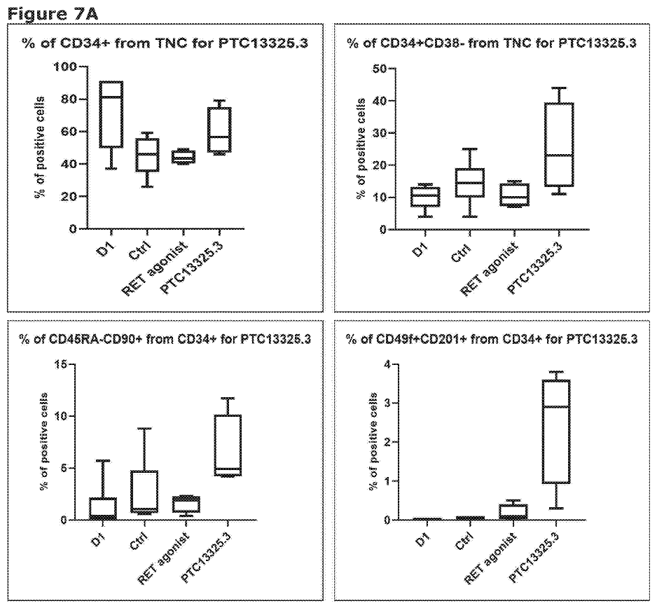

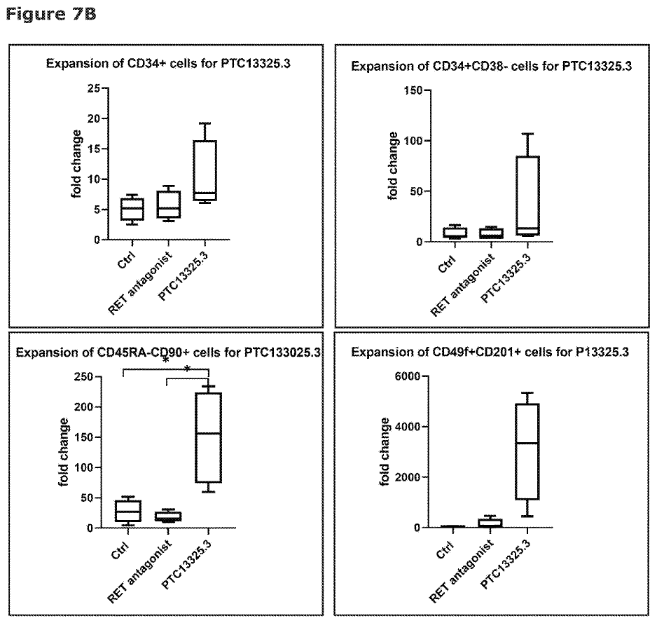

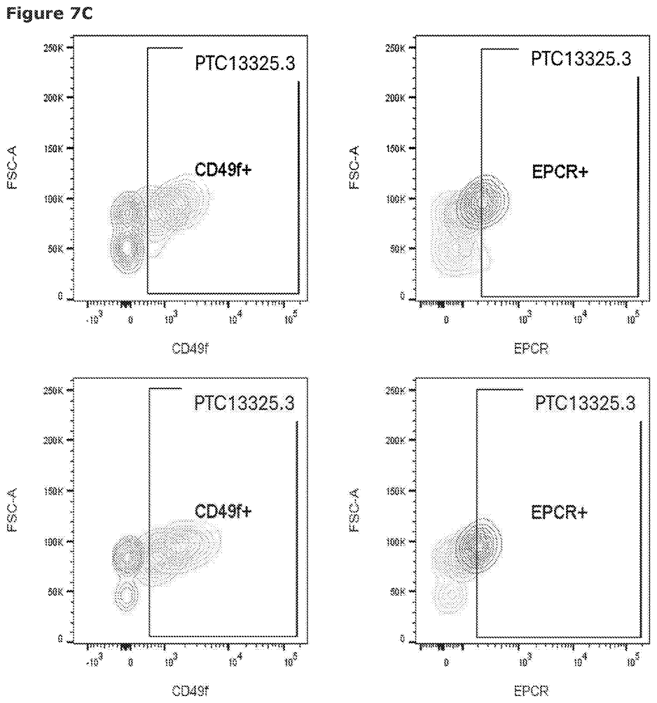

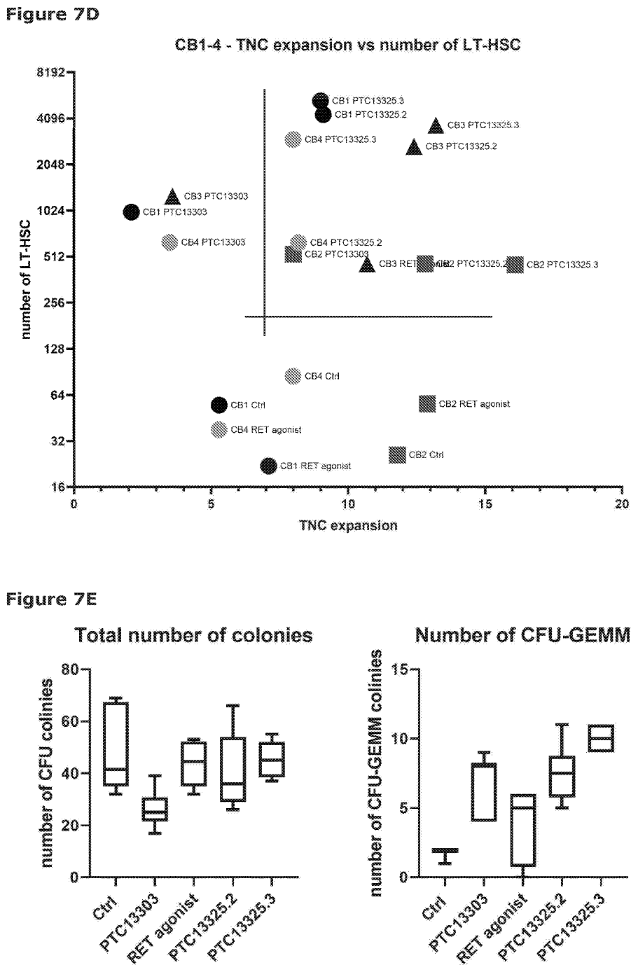

It was assumed that the first proliferative step of protocol PTC13325.2 could also be achieved by using other ex vivo amplifiers of hematopoietic cell numbers. Indeed, similar significant expansion in primitive HSC numbers when the cells were first treated in vitro by RET followed by HDAC inhibitor and WR (protocol PTC13325.3, FIG. 7A, B) was observed. In a similar manner, both CD90+ and CD90-cell populations showed increased expression of both CD49f and EPCR (FIG. 7C).

Independent on the donor response to the treatment, all 3 protocols produced tight clusters when plotted for the numbers of produced total nucleated cells (TNT) vs the numbers of cells in LT-HSC compartment. As a result, protocols PTC13325.2 and PTC13325.3 had similar effect that benefitted both total cell numbers and the highly engraftable cell compartment (FIG. 7D).

This last observation was supported by the increase in total and, most important, CFU-GEMM colony numbers produced by cells expanded ex vivo by protocols PTC13325.2 and PTC13325.3 as compared to protocol PTC13303 (FIG. 7E).

Therefore, the combination of the proliferative step with HDAC inhibitor is crucial for the expansion of primitive HSCs.

### DISCUSSION

The Examples demonstrate a method of UCB CD34 cell expansion that in a short period of time (7 days) expands CD34 cell numbers, increases the quantity of cells with phenotypic characteristics of Long-Term Repopulating Cells and produces superior engraftment results upon transplantation in a well-characterised animal model (pre-conditioned NSG mice).

The in vitro data indicate that the cells in PTC13325.1 and PTC13325.2 media expanded in higher quantities compared to cells grown in the Control basal media containing cytokines alone (TNT population). When placed in culture, HSCs usually differentiate and lose the expression of stem cell markers. In contrast, cells grown in the expansion media of Protocols PTC13303 (comparative), PTC13325.1 and PTC13325.2 had higher percent of CD34+ cells compared to the Control basal media alone. The most primitive (“true”, or non-committed) HSCs were phenotypically characterized for the expression of number of markers: CD34+/CD38−/CD45RA−/CD90+/CD49f+/CD201 (EPCR)+. It has been shown that such cells demonstrate long-term repopulation potential in animal xenograft models. It was shown that the percent of this population was high after expanding cells with PTC13303, and PTC13325.1 and PTC13325.2; and the total numbers of cells of this phenotype was significantly higher after the expansion with protocols PTC13325.1 and PTC13325.2 compared to PTC13303 or Control basal media alone. This is a significant finding as it indicates that cells expanded according to the protocols of the invention have better engraftment potential that those expanded according to methods of the prior art.

One of the surrogate in vitro tests to assess the functional potency of HSC is a colony-forming assay. The cells with stem cell or progenitor capabilities, when plated in semi-solid media with the number of cytokines, can divide and produce colonies with distinct morphological phenotypes, which helps to assess the amount and type of progenitor cells in the sample. The most primitive cells gave rise to the colonies with mixed GEMM morphology, indicating that these cells are higher in the hierarchy of the blood cell lineages compared to more committed progenitor cells. Our results of colony formation showed the dramatic increase in both total number of colonies and, specifically, the number of GEMM colonies after expansion of cells with PTC13325.1. These data correlate with the flow analysis and strongly indicate that LT-HSCs and hematopoietic progenitors produced by expansion with PTC13325.1 are functional cells. To note, cells expanded by PTC13303 produced higher numbers of GEMM colonies compared to the Control, however PTC13325.1 was superior in both numbers of functional progenitors and true HSCs.

To test whether cells expanded by PTC13325.1 were actually functional cells capable of hematopoietic reconstitution, the cells pooled from four donors and expanded by PTC13325.2 were transplanted into NSG mice. The engraftment was followed over time by analysing bone marrow aspirations (weeks 10 and 16) and end-point bone marrow reconstitution at week 18. The percent of human cells (by CD45 marker) was higher in PTC13325.2 treated at all time points and reached almost 100% in individual animals.

The PTC13325.2 differed from PTC13325.1 in the length of cell exposure to compounds SR1, SP600125, and IL-6 (see Materials and Methods); however, the exposure of cells to Scriptaid and WR-1065 remained the same in both time and length. In PTC13325.1, the exposure time to SR1, SP600125, and IL-6 was shorter as the cells were washed before adding HDAC/aminothiol.

To confirm that PTC13325.2 yielded an increased number of long-term HSCs, single cell transcriptomics analysis was performed to compare basal expansion media, PTC13303 and PTC13325.2. By applying unbiased clustering of integrated data and comparing the proportions of double-positive CD34/EPCR cells in every cluster, it could be seen that PTC13325.2 consistently increased the number of LT-HSCs compared to basal expansion media, in accordance with what was observed in the phenotype analysis and in vivo transplantation assays.

When protocols PTC13325.1 and PTC13325.2 were compared in vitro using UCB from 2 additional donors (FIG. 2E), it was observed that the Total Nucleated Cells (TNC) numbers were increased when cells were treated by both protocols. This increase was prominent when compared to the Control protocol (cytokines alone). The magnitude of the proliferative effect is donor-dependent, but the trend is always the same.

In ex vivo culture, hematopoietic stem cells (HSC, CD34+ cells) may proliferate, but rapidly lose their ‘stemness’ defined by the expression of markers such as CD34+ alone or in combination with CD38−(CD34+/CD38− are considered to be more primitive, less differentiated HSCs). It is important to define which type of cells underwent proliferation in the expanded cultures, and whether the ‘stemness’ characteristics of the cells were preserved or declined. For this we analysed the percentage of CD34+ and CD34+/CD38− cells inside the population of TNCs for the cells expanded by Control (cytokines alone) and protocols PTC13303, PTC13325.1 and PTC13325.2. We showed that cells treated by the Control protocol rapidly lose their stemness characteristics (CD34+ and, more significantly, CD34+/CD38− phenotype; Lin− is the Lineage negative population, that excludes most advanced progenitor cells), while their stemness is preserved following treatment with all 3 PTC protocols disclosed herein. We conclude, that PTC13325.1 and PTC13325.2 are capable of significantly increasing the proliferation of cells while preserving their stem cell phenotype. This is important for engraftment and therapeutic potential in conditions where the immune system is compromised.

There is an on-going research in the field of HSC to define a set of phenotypic markers, which reliably correlates with long-term engraftment potential of the cells upon transplantation. It is believed that long term engraftment is mediated by the most primitive population of HSC (LT-HSC), which are not yet committed to any of the blood cell lineages. Historically, the set of markers for this LT-HSC population was believed to be: Lin−/CD34+/CD38−/CD45RA−/CD90+/CD49f+, however recently a novel marker CD201 (EPCR) has emerged that is believed to be more predictive of engraftment potential. As previous in vitro experiments (including those disclosed in WO2020/084310 and WO2020/084310) were evaluated using CD49f as the surrogate marker for engraftment potential, we decided to compare our protocols using CD201 as a marker (in FIGS. 2E, 5, 6),

Single cell RNA-sequencing data was analysed, generated from CD34+/CD45RA− HSCs generated using protocols PTC13303, PTC13325.2 and Control, and freshly isolated from UCB without culturing (FIG. 5). The cell populations were characterised by unbiased clustering (FIG. 5B) and measuring the expression of cluster markers (FIG. 5C). Cell identity was also confirmed by the measurement of HSC and progenitor cell type signatures (FIG. 5D). Focusing on cell clusters that contain LT-HSCs, it was shown using this method that:
1. The percentage of LT-HSC is increased after treatment with the PTC protocols of the Examples compared to Control.
2. The percentage increase is more pronounced after treatment with PTC13325.2 rather than PTC13303.

Multiparametric flow cytometry is a proven method for immunophenotyping of HSCs and various subpopulations of progenitor cells. Using this technique to measure protein markers, an increase in the percentage of CD201+ cells and CD49f+ cells in the population of CD34+CD38−CD45RA−CD90+ cells after treatment with PTC13325.1 and PTC13325.2 protocols was observed, relative to Control media and PTC13303 (comparative) protocols (FIG. 2E (iv)).

Based on total number of nucleated cells (TNC) and percent of LT-HSC in the TNC population, the number of LT-HSC in expanded cultures were calculated. It was shown that in addition to the percentages of LT-HSC being higher in the protocols of the Invention PTC13325.1 and PTC13325.2 compared to the original protocol PTC13303, the total number of LT-HSC is also much higher when cells are expanded by PTC13325.1 and PTC13325.2 (FIG. 2E(iii)). The magnitude of these results is donor dependent, but the trend stays the same. This leads to the conclusion that the protocols of the invention result in expanded cell preparations that have superior engraftment potential.

The individual expanded cell populations were further phenotypically characterised after treatment with Control media or Protocols PTC13303, PTC13325.1 and PTC13325.2 and the results are shown in FIG. 2F. We used a well-established set of markers: CD34, CD38, and CD45RA; the expression of each of them indicates the commitment of HSC to the more mature hematopoietic progenitors. The acquisition of CD38 and CD45RA expression shows step-wise progression through the differentiation process, while CD38+/CD45RA+ cells are more differentiated than CD38+/CD45RA− or CD38−/CD45RA+ cells. The loss of CD34 expression indicates the more advanced differentiation steps. Thus, we analysed only the progenitor cells that retain the CD34 marker.

We show that cells treated by the Control protocol most rapidly accumulate the more differentiated phenotype expressing CD38+/CD45RA+ markers. The treatment with all three protocols PTC13303 (comparative), PTC13325.1 and PTC13325.2 decreases the proportion of differentiated cells; however, while the protocol PTC13303 (comparative) supports the undifferentiated status of cells (CD34+/CD38−/CD45RA−), the protocols of the invention PTC13325.1 and PTC13325.2 favour accumulation of progenitor cells expressing markers [CD38−/CD45RA+]. These phenotypic changes support rapid and stable hematopoietic reconstitution of the recipient by cells expanded with protocols PTC13325.1 and PTC13325.2. This is also supported by the widespread detection of progenitor signatures in the single cell dataset (FIG. 5D, 5E).

The protocols of the invention differ from the comparative example in that there is a pre-culturing step before the HDAC inhibitor and aminothiol are added. In the protocols of the invention, this pre-culturing step is carried out in the presence of SR1 and/or SP600125; however, it is believed that neither of these compounds is essential. It is believed that the act of having an additional culturing step of at least 48 hours before the addition of HDAC/aminothiol is required to achieve the results demonstrated herein.

1. A method to expand hematopoietic stem and progenitor cells (HSPC) wherein the method comprises:
i) obtaining an isolated population of HSPC;
ii) culturing the isolated population of HSPC for at least 48 hours to form a first cultured population;
iii) adding a histone deacetylase inhibitor (HDAC inhibitor) and further culturing the cells, to form a second cultured population; and
iv) adding an aminothiol compound to the second cultured population and further culturing the cells to form an expanded population of cells, wherein the aminothiol compound has the formula RNH(CₙH₂ₙ)NH(CₙH₂ₙ)SX, wherein R is hydrogen, an aryl, an acyl, or an alkyl group containing from 1 to 7 carbon atoms, each n has a value of from 2 to 6 and X is H or PO₃H₂; or a pharmaceutically acceptable salt thereof.

2. (canceled)

3. A method of treatment of a haematological disorder, immune disorder, metabolic disorder, or neurodegenerative disorder, the method comprising the steps of:
i) obtaining an isolated population of HSPC;
ii) culturing the isolated population of HSPC for at least 48 hours to form a first cultured population;
iii) adding a histone deacetylase inhibitor (HDAC inhibitor), and further culturing the cells to form a second cultured population;
iv) adding an aminothiol compound to the second cultured population and further culturing the cells to form an expanded population of cells, wherein the aminothiol compound has the formula
RNH(CₙH₂ₙ)NH(CₙH₂ₙ)SX, wherein R is hydrogen, an aryl, an acyl, or an alkyl group containing from 1 to 7 carbon atoms, each n has a value of from 2 to 6 and X is H or PO₃H₂; or a pharmaceutically acceptable salt thereof; and
v) administering the expanded population of cells to a subject.

4-5. (canceled)

6. The method according to claim 3, wherein the therapy or treatment comprises repopulating mammalian bone marrow.

7. The method according to claim 3, wherein the therapy or treatment is of acute myelogeneous leukemia, acute lymphoblastic leukemia, chronic lymphocytic leukemia, chronic myelogeneous leukemia, non-Hodgkin lymphoma, severe aplastic anemia, severe combined immunodeficiency or sickle cell disease.

8. The method according to claim 3, wherein the therapy or treatment is gene therapy.

9. (canceled)

10. The method according to claim 1, wherein step i) comprises selecting for cells which are CD133+ and/or CD34+.

11. (canceled)

12. The method according to claim 1, wherein;
a) step ii) comprises culturing the cells for about 48 hours to about 96 hours, preferably about 72 hours; and/or
(b) step iii) comprises culturing the cell for about 24 hours to about 72 hours, preferably about 48 to 72 hours; and/or
(c) step iv) comprises culturing the cell for about 16 hours to about 24 hours, preferably about 24 hours.

13-15. (canceled)

16. The method according to claim 1, wherein step ii) comprises culturing the cell in the presence of basal media.

17. The method according to claim 1, wherein step ii) further comprises culturing the cell in the presence of interleukin 6 (IL-6).

18. The method according to claim 1, wherein step ii) comprises culturing the cell in the presence of nicotinamide, pyrimido-indole derivatives such as UM171 and UM729, p38 MAPK inhibitors, Notch ligands, Wnt agonists, a JNK inhibitor, a cytokine, at least one RET agonist and/or an antagonist of the aryl hydrocarbon receptor.

19. (canceled)

20. The method according to claim 1, wherein between step ii) and step iii) the cells are washed, preferably wherein the cell culture medium at stage iii) onwards is substantially free of the JNK inhibitor and/or the antagonists of the aryl hydrocarbon.

21. The method according to claim 1, wherein the JNK inhibitor is SP600125 and/or the antagonist of the aryl hydrocarbon receptor is SR1.

22. (canceled)

23. The method according to claim 1, wherein step ii) comprises culturing the cells in the presence of at least one, preferably at least two RET agonist(s), optionally selected from GDNF, GFRα1, BT-13, Q525 and BT44.

24. (canceled)

25. The method according to claim 1, wherein the HDAC inhibitor is scriptaid or quisinostat.

26. (canceled)

27. The method according to claim 1, according to claims, wherein the aminothiol compound is amifostine:

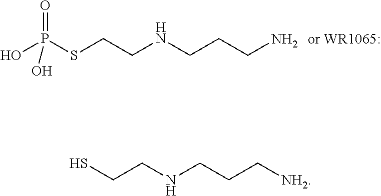 [SMILES: NCCCNCCSP(=O)(O)O ; NCCCNCCS]

28. (canceled)

29. The method according to claim 1, wherein the isolated population is obtained from umbilical cord blood, peripheral blood or bone marrow.

30-35. (canceled)

36. The method according to claim 1, wherein the expanded cells are enriched for hematopoietic stem cells (HSC) and/or long-term hematopoietic stem cells (LT-HSC).

37. The method according to claim 1, wherein the expanded cells are enriched for; Lin−, CD38−, CD34+, CD45RA−, CD90+, CD201+ and CD49f+.

38. The method according to claim 1, wherein the total cell expansion is between about 5-fold to about 20-fold, preferably about 10-fold to about 20-fold or the expansion of Lin−, CD38−, CD34+, CD45RA−, CD90+ and CD49f+ cells is about 500-fold.

39. (canceled)

40. The method according to claim 1, wherein the JNK inhibitor is used at a concentration of 0.01-50 μM, or the antagonist aryl hydrocarbon receptor is used at a concentration of 0.01-50 μM, or the IL-6 is used at a concentration of 0.01-50 μg/ml, preferably 0.1 μg/ml, or the at least one, preferably two RET agonist(s) is used at a concentration of 100 ng/ml, or the HDAC inhibitor is used at a concentration of 0.01-50 μM, or the aminothiol compound is used at a concentration of 50-500 μM.

41-48. (canceled)
